SEQUENCE LISTING (27 sequences)

SEQ ID NO: 1 - DNA, 22 bp, Rattus norvegicus
cagctgcagttccgtagaaaga

SEQ ID NO: 2 - DNA, 16 bp, Rattus norvegicus
gcgagcgacctacgcg

SEQ ID NO: 3 - DNA, 22 bp, Rattus norvegicus
tgtggcgtggtcacagccgaag

SEQ ID NO: 4 - DNA, 21 bp, Rattus norvegicus
aggaagaggcatccatcaacc

SEQ ID NO: 5 - DNA, 19 bp, Rattus norvegicus
gaaagtcaaggcccatggc

SEQ ID NO: 6 - DNA, 29 bp, Rattus norvegicus
caatgctatcaacaagtgtcccctgctga

SEQ ID NO: 7 - DNA, 19 bp, Rattus norvegicus
gccttgaccaccctggaaa

SEQ ID NO: 8 - DNA, 21 bp, Rattus norvegicus
gctccgctggaactgatacag

SEQ ID NO: 9 - DNA, 21 bp, Rattus norvegicus
taatcacggcgtttgcgcggt

SEQ ID NO: 10 - DNA, 21 bp, Rattus norvegicus
tggagagctttggtcttttgc

SEQ ID NO: 11 - DNA, 20 bp, Rattus norvegicus
gggtgagtcaccgcctacag

SEQ ID NO: 12 - DNA, 26 bp, Rattus norvegicus
tgagtagtgatgttcagcggagggcc

SEQ ID NO: 13 - DNA, 19 bp, Rattus norvegicus
cagctcaggtggcatggtg

SEQ ID NO: 14 - DNA, 21 bp, Rattus norvegicus
ggaagcacagatggcaatgtc

SEQ ID NO: 15 - DNA, 21 bp, Rattus norvegicus
ctcagacccatgcccctgcca

SEQ ID NO: 16 - DNA, 20 bp, Rattus norvegicus
gccaactcggtgtgcttatg

SEQ ID NO: 17 - DNA, 19 bp, Rattus norvegicus
gtacttgtcgggcatgggc

SEQ ID NO: 18 - DNA, 22 bp, Rattus norvegicus
ccacctgggccgtcctgatggt

SEQ ID NO: 19 - DNA, 21 bp, Rattus norvegicus
cagaggctgttcaccttggag

SEQ ID NO: 20 - DNA, 19 bp, Rattus norvegicus
agggtgtaaatctcgcgcc

SEQ ID NO: 21 - DNA, 22 bp, Rattus norvegicus
tgctgchhatatgggtacggcc

SEQ ID NO: 22 - DNA, 19 bp, Rattus norvegicus
acagatgtccaagccccca

SEQ ID NO: 23 - DNA, 20 bp, Rattus norvegicus
cgccatgtcagaccgtacaa

SEQ ID NO: 24 - DNA, 26 bp, Rattus norvegicus
acatctcagagcaagcgtcgacccag

SEQ ID NO: 25 - DNA, 19 bp, Rattus norvegicus
ctgcctgatctgctgcctc

SEQ ID NO: 26 - DNA, 20 bp, Rattus norvegicus
cacccctcctcaaagaacca

SEQ ID NO: 27 - DNA, 26 bp, Rattus norvegicus
accaggagaactggaggctgaccaca

(19) United States
(12) Patent Application Publication
(10) Pub. No.: US 2006/0172295 A1
(43) Pub. Date: Aug. 3, 2006

(54) Genes associated with schizophrenia adhd and bipolar disorders

(75) Inventors: Graeme Bilbe, Neuchatel (CH); Anu Kinnunen, Rheinfelden-Herten (DE); James Irvin Koenig, Corfton, MD (US)

Correspondence Address: BROWDY AND NEIMARK, P.L.L.C.;624 NINTH STREET, NW, SUITE 300, WASHINGTON, DC, 20001-5303, US

(73) Assignee: University of Maryland, Baltimore, MD (US)

(21) Appl. No.: 10/520,133

(22) PCT Filed: Jul. 10, 2003
(86) PCT No.: PCT/EP03/07491

§ 371 Date: Nov. 7, 2005

Related U.S. Application Data
(60) Provisional application No. 60/395,088, filed on Jul. 11, 2002.
(60) Provisional application No. 60/472,489, filed on May 22, 2003.

(51) Int. Cl. C12Q 1/68

(52) U.S. Cl. 435/6

(57) ABSTRACT

Disclosed are methods for diagnosing, monitoring the progression of, and treating schizophrenia, bipolar disorder, and/or ADHD based upon genes that are differentially expressed in said disorders at baseline, or at different timepoints following an acute stress exposure. Also disclosed are methods for identifying agents useful in the treatment of schizophrenia, bipolar disorder, and/or ADHD, methods for monitoring the efficacy of a treatment for schizophrenia, bipolar disorder, and/or ADHD, methods for preventing and treating schizophrenia, bipolar disorder, and/or ADHD, and an animal model for schizophrenia, bipolar disorder, and/or ADHD.

### FIELD OF THE INVENTION

The present invention relates generally to the field of neurological and physiological dysfunctions associated with schizophrenia, attention-deficit-hyperactivity disorder (ADHD) and bipolar disorder. The invention further relates to genes which, when varied in their normal expression pattern, are associated with schizophrenia, ADHD and bipolar disorder. Thus, the present invention relates to the novel use of known genes in schizophrenia, ADHD and bipolar disorder. The present invention also relates to methods for diagnosing and observing disease progression of schizophrenia, ADHD and bipolar disorder. The present invention further relates to methods for identifying agents useful for the suppression of schizophrenia, ADHD and bipolar disorder. The present invention further relates to the construction of animal models of schizophrenia, ADHD and bipolar disorder. Another aspect of the invention relates generally to neuropsychiatric disorders displaying a similar dysfunction of the hypothalamo-pituitary-adrenal axis as observed in the repeated variable prenatal stress animal preparation described herein, such as, schizophrenia, ADHD and bipolar disorder, where normalization of the function of the HPA-axis or acute stress response may have therapeutic benefits, or can be used to monitor disease progression and/or treatment response.

### BACKGROUND OF THE INVENTION

In many psychiatric disorders, stress is the major non-genetic factor contributing to the outbreak of the disease, to the manifestation or exacerbation of acute symptoms, to the recurrence or relapse after a period of remission and to the failure to respond to pharmacotherapy (e.g. Norman and Malla, Br J Psychiatry. 1993; 162:161-166; Peralta and Cuesta, Compr Psychiatry. 1994:198-204; Jansen et al., Psychopharmacology (Berl). 2000; 149:319-325.)

Schizophrenia is a devastating neuropsychiatric disorder with a 1% worldwide prevalence (Carpenter W T Jr and Buchanan R W, N Engl J Med. 330:681-690 (1994); Hyman S E, Biol Psychiatry. 47: 1-7 (2000)). Its etiology is multifactorial with a complex genetic component and several disease loci identified so far (for review, see Pulver, Biol Psychiatry. 47:221-30. (2000)). A high discordance among twins, and epidemiological studies suggest the involvement of environmental factors in the origin of the disease (Weinberger, Lancet 346: 552-557 (1995); Hyman, Bull World Health Organ. 78:455-63 (2000), Tsuang et al., Br J Psychiatry (Suppl). 40:18-24 (2001)). One of the most frequently reported environmental risk factors for developing schizophrenia is a chronic maternal stress in a form or another: Prenatal loss of the father (Huttunen and Niskanen, Arch Gen Psychiatry. 35:429-431. (1978)), influenza (Mednick et al., Arch Gen Psychiatry, 45:189-192 (1988)), war (van Os and Selten, Br J Psychiatry, 172:324-326 (1998)) or natural disaster (Selten, Schizophr Res. 35:243-245 (1999); Kinney et al., J Abnorm Psychol., 108:359-362 (1999); Watson et al., Dev Psychopathol. 11:457-466 (1999)). On the other hand, chronic maternal stress during pregnancy may increase the likelihood for preterm birth, and lower birth weight and anoxia which by themselves have been found to be riskfactors for schizophrenia (Jones et al., Am. J. Psychiatry 155:355-364 (1998); Hultman et al., BMJ, 318:421-426 (1999)). Gestational stress has also been found to be associated with higher incidence of attention-deficit-hyperactivity disorder (ADHD; Clements et al., Ga. Educ. Res. 91:1-14 (1992)), bipolar disorder (Brown et al., Am J Psychiatry., 157:190-195 (2000)) and unipolar depression (Watson et al., Dev Psychopathol., 11:457-466 (1999); Brown et al., Am J Psychiatry., 157:190-195 (2000)). The nature of the behavioral abnormality caused by maternal stress is probably determined by the time and duration of the stress in relation to the stage of development of particular neuronal system. The timing of the stressful event during the second trimester, in contrast to the first or the third, seems to be critical for schizophrenia (Imamura, Acta Psychiatr Scand 100:344-349 (1999); Myhuman et al., Br J Psychiatry 169:637-40 (1996)) and could also account for the negative outcomes in some studies (Crow, Br. J. Psychiatry 164:588-592 (1994); Westergaard et al., Arch Gen Psychiatry 56:993-998 (1999)). Assessment of maternal stress as the cause of the behavioral pathology in the epidemiological studies is often confounded by factors such as recall bias, concomitant drug intake, poor nutrition, pregnancy symptoms, ill health and/or small size of the study population. To isolate gestational stress from other possibly contributing factors well-controlled studies in experimental animals have been undertaken mostly in rats and in non-human primates (for review, see Weinstock, Progr. Neurobiol. 65: 427-451 (2001)). Maternal stress in rats and monkeys has been shown to result in decreased social interaction of the prenatally stressed offspring (Ward and Stehm, Physiol. Behav. 50:601-605 (1991); Clarke and Schneider, Dev. Psychobiol. 26:293-304 (1993)) which is in accordance with human findings (Meijer, Acta Psychiatr. Scand. 72:505-511 (1985); Done et al., BMJ 309:699-703 (1994)). Furthermore, in adulthood, most prenatally stressed rat preparations show increased anxiogenic behaviour (Thompson, Science 15:698-699 (1957); Wakshlak and Weinstock, Physiol. Behav. 48:289-292 (1990); Poltyrev et al., Dev. Psychobiol. 29:453-462 (1996); Vallée et al., J. Neurosci. 17, 2626-2636 (1997)) and depressive behaviour as measured by increased immobility time in the forced swim test (Alonso et al., Physiol. Behav. 50:511-517 (1991); Drago et al., Eur Neuropsychopharmacol. 9:239-45 (1999) and Weinstock, In: Myslobodsky, M and Weiner, I. (Eds.) Contemporary Issues in Modeling Psychopthology. Kluwer, Dordrecht, pp. 45-54 (2000)), increased anhedonia (Keshet and Weinstock, Pharmacol Biochem Behav. 50:413-419 (1995)) and altered sleep-wake cycle (decreased REM-sleep latency, prolongation of the first REM-sleep episode and decrease in slow-wave-sleep; Rao et al., Prog. Neuropsychopharmacol Biol Psychiatry. 23: 929-939 (1999)). However, no impairment of prepulse inhibition (PPI), as a measure of abnormality of sensory gating (inability to filter out the flow of excessive sensory information reaching consciousness) has so far been observed in prenatally stressed animals. On the contrary, Lehmann et al. (Behav Brain Res. 107: 133-44 (2000)) reported an increase in the PPI in rats that during their gestation had been exposed to prenatal restraint stress (3×/week). Recently, Koenig et al. (Schizophrenia Res. 49(suppl):92 (2001)) have demonstrated that prenatal stress during the second week of gestation failed to alter prepulse inhibition responses, while exposure to stress during the third week of gestation (corresponding to the second trimester in humans in terms of brain development, Bayer et al., Neurotoxicology 14:83-144 (1993)) resulted in disruption of sensorimotor gating as measured by PPI. Isolation rearing, which appears to be a social stressor, also leads to abnormal prepulse inhibition (Varty and Geyer, Behav. Neuroscience 112:1-8 (1998)). Cognitive deficits, disrupted hippocampal anatomy and deficits in sensorimotor gating, i.e. PPI and P50, are features commonly observed in schizophrenic patients (Flaum et al., J. Psychiatr. Res. 29:261-276 (1995); Bilder et al., Schizophr. Res. 17:47-58 (1995); Freedman et al., Proc. Natl. Acad. Sci. USA 94:587-592 (1997); Clementz et al., Am. J. Psychiatry 155:1691-1694 (1998); Velakoulis et al., Arch. Gen. Psychiatry 56:133-141 (1999); Stefanis et al., Biol. Psychiatry 46:697-702 (1999); Gur et al., Arch. Gen. Psychiatry 57:769-775 (2000); Kupferberg and Heckers, Curr. Opin. Neurobiol. 10:205-210 (2000); Walder et al., Biol. Psychiatry 48:1121-1132 (2000)). Lemaire et al. (Proc. Natl. Acad. Sci. USA 97:11032-11037 (2000)) provided evidence showing maternal stress in rats resulted in the offspring having smaller hippocampi and spatial learning deficits due to impairment in the learning-associated neurogenesis in the dentate gyrus.

Prenatal stress increases maternal corticotropin and corticosterone concentrations. Corticosterone can readily penetrate the fetal brain (Zarrow et al., 1970) and interact with specific glucocorticoid receptors that are present during the last week of gestation in the rat (Meaney et al., 1985; Cintra et al., 1993). Glucocorticoid receptors are nuclear hormone receptors which function as ligand-activated transcription factors directly mediating transactivation of target genes by binding sequence specific recognition elements (glucocorticoid response elements; Whitfield et al., 1999). Glucocorticoid receptors are also known to interact with multiple transcription factors, such as c-jun, nuclear factor-κB, the TFIID complex, STAT5, and co-activators known to modulate the function of these signaling molecules (Jenkins et al., 2001; Yudt and Cidlowski, 2002). Prenatal stress also increases maternal and foetal catecholamine release (Morishima et al., 1978; Roehde et al., 1989), maternal oxytocin and opiold peptides of which β-endorphin is able to cross the placenta (Sandman and Kastin, 1981; Neumann et al., 1998). Molecular pathways leading from prenatal stress to the neuroendocrinological, behavioral, molecular and neurochemical changes in the adult remain poorly understood. In addition to complex glucocorticoid regulated mechanisms, it is likely that the plasticity of the developing brain monoaminergic system participates in these changes (Weinstock, 2001; Welberg and Seckl, 2001, Seckl, 2001).

It has been shown repeatedly that exposure to stress in utero reprograms the adult hypothalamo-pituitary-adrenal (HPA)-axis resulting in greater and prolonged elevation of plasma ACTH and/or corticosterone after acute stress (Peters, Pharmacol. Biochem. Behav. 17:721-726 (1982); Henry, J. Neuroendocrinol. 6:341-345 (1994); McCormic, Brain Res Dev Brain Res. 84:55-61 (1995); Barbazanges et al., J. Neurosci. 16:3943-3949 (1996; Vallée et al., J. Neurosci. 17, 2626-2636 (1997); Dugovic et al., J Neurosci. 19:8656-64 (1999)) which may be due to a decrease in type I and type II glucocorticoid receptors in the hippocampus after prenatal stress (Henry et al., J. Neuroendocrinol. 6:341-345 (1994), Koehl et al., J. Neurobiol. 40:302-315 (1999)). The disturbance of the HPA-axis is consistent with schizophrenia (Yeragani, Can. J. Psychiatry 35:128-132 (1990); Goldman et al., Am. J. Psychiatry 150:653-655 (1993); Elman et al., Am. J. Psychiatry 155:979-981 (1998); Newcomer et al., Biol. Psychiatry 29:855-864 (1998)), generalized anxiety disorder and depression in humans (Arborelius et al., J Endocrinol. 160:1-12 (1999)). Schizophrenic patients have been also shown to have fewer glucocorticoid receptors in postmortem schizophrenic brain tissue (Knable et al., Schizophrenia Res. 49(Suppl):53 (2001)).

Gestational stress has been shown to cause changes in brain morphology such as a reduction in hippocampal synapses (Hayashi et al., Int J Dev Neurosci. 16: 209-216 (1998)) which may be partially due to the reduced neuronal number in hippocampus as a result of maternal stress as shown by Lemaire et al. (Proc. Natl. Acad. Sci. USA 97:11032-11037 (2000)). Also, lower levels of N-acetyl aspartate (NAA) which is used as a marker for neuronal metabolism have been reported in the left prefrontal cortex (PFC) of prenatally stressed rats (Poland et al., J Psychiatr Res. 33: 41-51 (1999)) which has also be shown for untreated schizophrenic PFC and hippocampus (Buckley et al., Biol Psychiatry. 36:792-800 (1994); Bertolino et al., Biol Psychiatry. 43:641-8 (1998)).

Glucocorticoid hormones or stress have been shown to influence neurotransmission at many levels. For the brain dopaminergic system, these effects appear to be mediated by glucocorticoid receptors that are expressed in many dopamine (DA)-containing neurons in the brain (Harfstrand et al., Proc. Natl. Acad. Sci. USA 83:9779-9783 (1986); Cintra et al., Neuroendocrinology 57:1133-1147 (1993)). Glucocorticoids enhance DA metabolism (Tanganelli et al., J. Neural Transm. Gen. Sect. 81:183-194 (1990); Takahashi et al., Brain Res. 574:131-137 (1992); Diaz et al., Neuroscience 81:129-140 (1995); Barrot et al., Eur. J. Neurosci. 12:973-979 (2000), Eur. J. Neurosci. 13:812-818 (2001)) in the striatum and increase DA release in the nucleus accumbens (Piazza et al., Proc. Natl. Acad. Sci. USA 93:8716-8720 (1996); Barrot et al., Eur. J. Neurosci. 12:973-979 (2000)), although also decreased DA metabolism in the nucleus accumbens has been reported (Alonso et al., Pharmacol. Biochem. Behav. 49:353-358 (1994)). Furthermore, increased D2 receptor binding has been reported in the nucleus accumbens (Henry et al., Brain Res. 685:179-186 (1995)). In human subjects, glucocorticoid administration increases plasma HVA levels while inducing psychotic symptoms (Wolkowitz, Psychoneuroendocrinology 19:233-255 (1994)). In schizophrenic patients, augmentation in the dopaminergic state has been beautifully demonstrated by Laruelle et al. (Proc. Natl. Acad. Sci. USA 93:9235-9240 (1996), Biol. Psychiatry 46:56-72 (1999)).

In addition to changes in the dopaminergic system, exposure to glucocorticoids causes an increase in the turnover of norepinephrine in the cortex and locus coeruleus (Takahashi et al., Brain Res. 574:131-137 (1992)). It also increases serotonin synthesis in fetal brain (Peters, Pharmacol. Biochem. Behav. 25:873-877 (1986), Pharmacol. Biochem. Behav. 31:839-843 (1988), Pharmacol. Biochem. Behav. 35:943-947 (1990)), as well as concentrations of the brainstem serotonin transporter in rats (Slotkin et al., Brain Res. Dev. Brain Res. 93:155-161 (1996)). During rat brain development, glucocorticoid receptors first appear on embryonic day (E) 13 (Cintra et al., Neuroendocrinology 57:1133-1147 (1993)) which coincides the developmental phase of all the monoaminergic neurotransmitter systems. Therefore, early exposure to glucocorticoids also modifies the development of neurotransmitter systems proposed to be involved in the pathophysiology of schizophrenia, including norepinephrine, serotonin, dopamine and GABA (Davis et al., Am J Psychiatry 148:1474-1486 (1991); Roth and Meltzer, In Bloom F E and Kupfer D J (Eds), Psychopharmacology: The fourth generation of progress, New York, Raven Press, pp 1215-1227 (1995); Muneoka et al., Am. J. Physiol. 273:R1669-1675 (1997); Slotkin et al., Brain Res. Dev. Brain Res. 93:155-161 (1996); Diaz et al., Neuroscience 66:467-473. (1995); Neuroscience 81:129-140 (1997); Lewis, Brain Res Brain Res Rev. 31:270-6. (2000).

While effects of prenatal stress on the HPA axis and on some of the neurotransmitter systems are relatively well known, and the concept of prenatal programming has been a long-standing topic in neuroendocrinology research (for reviews, see e.g. Weinstock, Progr. Neurobiol. (2001); Weldberg and Seckl, J. Neuroendocrinolol., 13:113-128 (2001)) the molecular mechanisms resulting in and from the reprogramming remain poorly understood and, changes at the global gene expression level have not been studied previously. The hypothesis (see Koenig et al., Neuropsychopharmacol. 27: 309-318 (2002)) that prenatal hypercortisolemia coinciding a critical neurodevelopmental period in the embryo increases the risk of schizophrenia and other disorders with neurodevelopmental component (such as bipolar disorder and ADHD) has been shown to be biologically plausible as analogous changes can be observed in animals under controlled circumstances. Prenatal stress provides a testable preclinical and clinical model for underlying molecular pathophysiology and cure of the above mentioned human disorders.

Antipsychotic drugs were identified in the 1950's, and these drugs were found to produce a dramatic improvement in the psychotic phase of the illness. Reserpine was the first of these drugs to be used and was followed by typical antipsychotic drugs including phenothiazines, the butyrophenones, and the thioxanthenes. A new group of therapeutic drugs, typified by clozapine, has been developed and were referred to as “atypical” antipsychotics. Haloperidol has been employed extensively in the treatment of schizophrenia and is one of the currently preferred options for treatment. When these drugs are taken over the course of at least several weeks, they mitigate or eliminate delusions, hallucinations, and some types of disordered thinking. Maintenance of a patient on these drugs reduces the rate of relapse. Since there is no way of determining if an individual is susceptible to schizophrenia, it is currently unknown if these antipsychotic compounds are useful in the prophylactic treatment of schizophrenia.

Signal transduction is the general process by which cells respond to extracellular signals (e.g. neurotransmitters) through a cascade of biochemical reactions. The first step in this process is the binding of a signaling molecule to a cell membrane receptor that typically leads to the inhibition or activation of an intracellular enzyme. This type of process regulates many cell functions including cell proliferation, differentiation, and gene transcription (WO 01/26622). One important mechanism by which signal transduction occurs is through G-proteins. Receptors on the cell surface are coupled intracellularly to a G-protein that becomes activated, when the receptor is occupied by an agonist, by binding to the molecule GTP. G-proteins may influence a large number of processes including voltage-activated calcium channels, adenylate cyclase, and phospholipase C.

There is a need in the art for the identification of schizophrenia, ADHD and bipolar disorder associated genes. Identification of such genes would provide a fundamental understanding of the disease process from which a number of clinically important applications would arise. Said genes identified may lead to the development of therapeutics (small molecule drugs, antisense molecules, antibody molecules) directly targeted to the gene or protein product of the gene, or may target the biochemical pathway of which the protein product is a part at an upstream or downstream location if the development of such drugs is easier than directly targeting the gene or its protein product. Polynucleotide sequences comprising the gene, sequence variants thereof and protein products thereof may be used to develop a clinical diagnostic test for schizophrenia, ADHD and bipolar disorder and for the identification of individuals at high risk for the development of schizophrenia, ADHD and bipolar disorder. The results of such tests may also have prognostic value and may be used to predict patients who respond to and those who do not respond to drug therapy.

Prefrontal cortex, which is part of the frontal pole, is a target for glucocorticoids involved in stress response (Meaney M J and Aitken D H, Brain Res. 328:176-180. (1985)), it regulates the negative glucocorticoid feedback of the HPA-axis (Diorio D. et al., J. Neurosci. 13:3839-3847 (1993); Moghaddam B., Biol Psychiatry. 51, 775-787. (2001)), it shows neurochemical and neuroanatomical changes in response to stress (Wellman C., J. Neurobiol. 49:245-253 (2001)), and mediates many of the behaviors that are altered by chronic stress or corticosterone treatment, for example memory and learning. Identification of the abnormal stress response pattern at gene expression level in the frontal pole is relevant with regard to any stress provoked or stress-deteriorating human condition where frontal cortical structures play a role as parts of circuitry, such as depression, bipolar disorder, schizophrenia, anxiety, panic disorder, generalized anxiety disorder, post-traumatic stress syndrome, bipolar disorder, and addiction, and has got a potential for identification of novel therapeutic approaches that aim at normalizing the abnormal stress response in order to alleviate the resulting conditions, or as biomarkers for therapeutic interventions that also normalize the abnormal stress response.

### SUMMARY OF THE INVENTION

In a first aspect, the invention provides the use of isolated DNAs in the prevention and treatment of schizophrenia, ADHD and/or bipolar disorder comprising the nucleotide sequences referenced in Tables 1 and 2. Also provided are the use of isolated DNA's that comprise nucleic acid sequences in said indications that hybridize under high stringency conditions to the isolated DNA referenced in Tables 1 and 2 (herein referred to as the “Genes of the invention”). Also provided are the use of said nucleic acid sequences comprising at least about 15 bases, preferably at least about 20 bases, more preferably a nucleic acid sequence comprising about 30 contiguous bases the nucleic acid sequence referenced in Tables 1 and 2.

In a second aspect, the invention provides the use of isolated polypeptides in the prevention and treatment of schizophrenia, ADHD and/or bipolar disorder with the amino acid sequences encoded by the Genes of the invention. Such polypeptides, or fragments thereof, are found e.g. in the prefrontal pole and/or hypothalamus of sufferers of schizophrenia, ADHD and/or bipolar disorder in a different amount than in the same tissues of individuals without schizophrenia, ADHD and/or bipolar disorder.

A third aspect of the present invention encompasses a method for the diagnosis of schizophrenia, ADHD and/or bipolar disorder in a human which includes detecting the elevated and/or lowered transcription of messenger RNA transcribed from the Genes of the invention, where such altered transcription is diagnostic of the human's affliction with schizophrenia, ADHD and/or bipolar disorder. Another embodiment of the assay aspect of the invention provides a method for the diagnosis of schizophrenia, ADHD and/or bipolar disorder in a human which requires measuring the amount of a polypeptide that include or are the polypeptides which are encoded by the Genes of the invention, e.g. in the frontal pole and/or hypothalamus from a human, where the presence of an elevated or lowered amount of the polypeptide or fragments thereof, relative to the amount of the polypeptide or fragments thereof in e.g. in frontal pole and/or hypothalamus from a healthy human, is diagnostic of the human's suffering from schizophrenia, ADHD and/or bipolar disorder. A yet further aspect of the present invention is a method of determining susceptibility to schizophrenia, ADHD and/or bipolar disorder comprising obtaining from a patient to be tested for susceptibility to schizophrenia, ADHD and/or bipolar disorder a sample of tissue, measuring levels of the polypeptides encoded by the Genes of the invention in said sample, and determining if there is an elevated or lowered level of the polypeptides encoded by the Genes of the invention in the sample. Another aspect of the present invention is a kit for diagnosing schizophrenia, ADHD and/or bipolar disorder in a patient, said kit comprising antibodies to the polypeptides encoded by the Genes of the invention, and a detector for ascertaining whether said antibodies bind to the polypeptides encoded by the Genes of the invention in a sample. Another aspect of the present invention is a kit for diagnosing schizophrenia, ADHD and/or bipolar disorder in a patient, said kit comprising a detection tool to measure transcript levels of the Genes of the invention in a patient, and of a standard to ascertain altered levels of transcript of the Genes of the invention in the patient.

In another aspect, the invention is directed to methods for the identification of molecules that can bind to the polypeptides encoded by the Genes of the invention and/or modulate, i.e. activate or inhibit the activity of the polypeptides encoded by the Genes of the invention or molecules that can bind to nucleic acid sequences of the Genes of the invention that modulate the transcription or translation of said Genes. Such methods are disclosed in, e.g., U.S. Pat. Nos. 5,541,070; 5,567,317; 5,593,853; 5,670,326; 5,679,582; 5,856,083; 5,858,657; 5,866,341; 5,876,946; 5,989,814; 6,010,861; 6,020,141; 6,030,779; and 6,043,024, all of which are incorporated by reference herein in their entirety. Molecules identified by such methods also fall within the scope of the present invention.

Yet another aspect of the present invention is a method of treating schizophrenia, ADHD and/or bipolar disorder said method comprising measuring levels of the Polypeptides of the invention or levels of mRNA of the Genes of invention in a patient, and altering said polypeptide levels to provide the patient with an improved psychiatric function. In accordance with one embodiment of this aspect of the invention there are provided antisense polynucleotides that regulate transcription of the Genes of the invention; in another embodiment, double stranded RNA is provided that can regulate the transcription of the Genes of the invention.

An additional aspect of the present invention is an animal model for schizophrenia, ADHD and/or bipolar disorder where a repeated variable maternal stress was applied on Sprague Dawley rats during the gestational days 14-22, coinciding with the second trimester in humans, as opposed to repeated administration of the same stress to the pregnant animal.

A still additional aspect of the invention is the use of the genes identified in Tables 5, 6 and 7 in treating schizophrenia, ADHD and/or bipolar disorder and/or other stress aggravated psychiatric condition where dysregulation of the normal HPA axis function is a clinical finding (dysregulation of the HPA axis in this instance is defined as prolongation of the corticosterone response after acute stress or as insensitivity to glucocorticoid negative feedback. In humans, this corresponds to an “escape” from dexamethasone induced suppression of the HPA axis or abnormal elevation of diurnal cortisol in the a.m. or p.m. in patients); methods comprising measuring levels of the Polypeptides of the invention or levels of mRNA of the Genes of invention in a patient, and altering said polypeptide levels to provide the patient with an improved psychiatric function. Another dimension is the use of genes identified in Tables 5-7 as biomarkers of the disease progression or treatment response.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### DESCRIPTION OF THE INVENTION

All patent applications, patents and literature references cited herein are hereby incorporated by reference in their entirety.

In practicing the present invention, many conventional techniques in molecular biology, microbiology, and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The present invention relates to the identification of genes that are expressed at higher or lower levels in the frontal lobe and/or hypothalamus in a patient with schizophrenia, ADHD and/or bipolar disorder, than as in the same tissue in a healthy individual. By virtue of their low or high expression in said normal body tissues including normal frontal lobe and/or hypothalmic tissues and their high or low expression in said tissues in a patient with schizophrenia, ADHD and/or bipolar disorder these genes can be utilized in the diagnosis, disease management, screening for agonists and antagonists of the Genes of the invention, disease prevention and treatment, and/or post-treatment follow-up of men at risk for, with, or at risk for recurrence of said disorders which include, but are not limited to, schizophrenia, ADHD and/or bipolar disorder. These Genes of the invention include those listed in Tables 1 and 2 and are for the first time, as to our knowledge, associated with schizophrenia, ADHD and/or bipolar disorder and those listed in Tables 5, 6 and 7 for the first time associated with either normal or abnormal stress response related to disruption of HPA-axis. The complete sequences of the all the Genes of the invention are available from public databases (NCBI) using the accession numbers shown in Table 1, 2, 5, 6, and 7.

Although stress is commonly used to exacerbate behavioral abnormalities in psychiatric animal models (e.g. Piazza and Le Moal, Trends Pharmacol Sci. 1998; 19:67-74) abnormal stress responses in general, have not been incorporated in the animal models themselves. In an attempt to search for a novel heuristic animal model for schizophrenia we have incorporate stress in a form of repeated variable prenatal stress to pregnant Sprague-Dawley rats in a randomized manner during the last week of pregnancy (Koenig et al., 2002, Society for Neuroscience Abstracts, 495.6; Kinnunen et al., 2002, Society for Neuroscience Abstracts, 495.7). This in terms of brain development, corresponds to the second trimester in humans (Bayer et al., 1993). The offspring is left undisturbed until weaning and behavioral testing on days 35 and 56. The locomotor response to amphetamine and phencyclidine (PCP) is enhanced in the prenatally stressed offspring on postnatal day 56 but not on postnatal day 35. The prenatally stressed male offspring displayed a disruption of sensorimotor gating as measured by prepulse inhibition (PPI) and in sensory gating assessed using the N40 response and a disruption in the normal corticosteroid negative feedback are commonly observed deficits in schizophrenic patients. Also the emergence of the behavioral deficits postpubertally is in accordance with schizophrenia.

Using said microarray analysis approach genes were identified that in the frontal pole are differentially regulated following an acute stress exposure. Based on the literature, some of these genes are known to be regulated by acute stress or corticosterone, e.g. c-fos (e.g. Martinez et al., Stress 2002; 5:3-13), sgk (Webster et al., Mol. Cell. Biol., 1993; 13:2031-2040; Naray-Fejes-Toth et al., J. Steroid Biochem. Mol. Biol., 2000, 75:51-56) and CART (Vrang et al., Brain Res., 2003, 965: 45-50). The gene expression response that is occurring in the normal control animals and is partially overlapping with the acute stress response in the prenatally stressed animals, provides a pattern for “normal stress response” in adult Sprague-Dawley male rats. Furthermore, by the present invention genes were identified that are differentially regulated by acute stress in the prenatally stressed animals only, or are differentially regulated by acute stress following a different time course than in the normal controls. Those genes that are differentially regulated after the acute stress in the prenatally stressed therefore demarcates the gene expression component of “abnormal stress response” in adult Sprague-Dawley male rats.

Any selection of at least one of the genes listed in Tables 1, 2, and/or 3 can be utilized as a marker and/or therapeutic target for Schizophrenia, bipolar disorder, and/or ADHD with the proviso that if expression of only one gene is detected that the gene is not one of the genes of Table 3. In some embodiments, if expression of only one gene is detected the gene is not one of Table 3 which are already known to be linked with schizophrenia, bipolar disorder, ADHD. In preferred embodiments, any selection of at least one of the Genes of the invention can be utilized as a marker and/or therapeutic target for schizophrenia, bipolar disorder, ADHD. In particularly useful embodiments, the genes in Tables 1 and/or 2 can be utilized as a therapeutic target for schizophrenia, bipolar disorder, ADHD. In particularly useful embodiments, a plurality of these genes or at least two or more of the genes listed in Table 1, 2, and/or 3 and Table 5, 6 and/or 7 can be selected and their expression monitored simultaneously to provide expression profiles for use in various aspects. For example, expression profiles of the genes provide valuable molecular tools for rapidly diagnosing and monitoring the progression of schizophrenia, bipolar disorder, and/or ADHD, and for evaluating drug efficacy. Changes in the expression profile from a baseline profile can be used as an indication of such effects. Accordingly, the invention provides methods for screening a subject for (diagnostic) schizophrenia, bipolar disorder, and/or ADHD or at risk of developing (prognostic) schizophrenia, bipolar disorder, and/or ADHD, methods for monitoring the progression of schizophrenia, bipolar disorder, and/or ADHD in a subject, methods for the identification of agents that are useful in treating a subject having or at risk of having schizophrenia, bipolar disorder, and/or ADHD, methods of treating a subject having or at risk of having schizophrenia, bipolar disorder, and/or ADHD, methods for monitoring the efficacy of certain drug treatments for schizophrenia, bipolar disorder, and/or ADHD, and vectors for schizophrenia, bipolar disorder, and/or ADHD-specific replication.

The most differentially expressed genes are identified in Tables 1, 2 and/or 3. Particularly preferred genes listed in Table 1 and/or 2 include genes involved with synaptic vesicle endo- and exocytosis, genes which are found to be in the postsynaptic density, some of the NMDA, AMPA- and GABA receptor subunits.

The method for screening a subject for schizophrenia, bipolar disorder, and/or ADHD or at risk of developing schizophrenia, bipolar disorder, and/or ADHD comprises:
a) detecting a level of expression of at least one of the genes identified in Tables 1, 2, and/or 3 in a sample of brain tissue, e.g. frontal pole and/or hypothalamus obtained from a mammal, e.g. human or animal model to provide a first value; and
b) comparing the first value with a level of expression of the at least one gene identified in Tables 1, 2, and/or 3 in a sample of fluid or tissue as above obtained from a subject free of schizophrenia, bipolar disorder, and/or ADHD, wherein a greater or lesser, expression level in the subject sample, compared to the sample from the subject free of schizophrenia, bipolar disorder, and/or ADHD, is indicative of the subject having schizophrenia, bipolar disorder, and/or ADHD or at risk of developing schizophrenia, bipolar disorder, and/or ADHD.

The brain tissue sample (e.g. from the frontal pole and/or hypothalamus) can be obtained from the subject, a human or animal model, by known surgical methods, e.g., surgical resection or needle biopsy. When assessing the level of expression of the gene by measuring the level of mRNA as described below, it is preferable to obtain a brain tissue sample, e.g. frontal pole and/or hypothalamus, from the subject. The sample taken from the subject free from schizophrenia, bipolar disorder, and/or ADHD can be a sample of normal brain tissue (e.g. frontal pole and/or hypothalamus) or bodily fluid from the same individual or from another individual. For example, in examination of a suspected disorder such as schizophrenia, bipolar disorder, and/or ADHD, the sample from the subject free from schizophrenia, bipolar disorder, and/or ADHD (disease-free subject) can be a sample of normal brain cells from the individual suspected of having schizophrenia, bipolar disorder, and/or ADHD. The sample obtained from the disease-free subject can be obtained at the same time as the sample obtained from the subject, or can be a pre-established control for which expression of the gene was determined at an earlier time. The level of expression of the gene in the sample obtained from the disease-free subject is determined and quantitated using the same approach as used for the sample obtained from the subject. The level of expression of at least one of the disclosed genes in the samples obtained from the subject and disease-free subject can be detected by measuring either the level of mRNA corresponding to the gene, the protein encoded by the gene or a fragment of the protein, e.g., the catalytic domain. In the methods of the invention, the level of expression of one of the disclosed genes in a diseased tissue preferably differs from the level of expression of the gene in a non-diseased tissue by a statistically significant amount. In presently preferred embodiments, at least about a 1.5-fold difference in expression levels is observed. In some embodiments, the expression levels of a gene differ by at least about 2-, 3-, 4-, 5-, 10- or 100-fold or more in the diseased tissue compared to the non-diseased tissue. The level of expression of at least one of the genes disclosed in Table 1 is determined in the methods of the invention. It is sometimes desirable to determine the level of expression of 2, 3, 5, 10, 20, or more of the disclosed genes. RNA can be isolated from the samples by methods well known to those skilled in the art as described, e.g., in Ausubel et al., Current Protocols in Molecular Biology, Vol. 1, pp. 4.1.1-4.2.9 and 4.5.1-4.5.3, John Wiley & Sons, Inc. (1996). Methods for detecting the level of expression of mRNA are well known in the art and include, but are not limited to, northern blotting, reverse transcription PCR, real time quantitative PCR and other hybridization methods. A particularly useful method for detecting the level of mRNA transcripts obtained from a plurality of the disclosed genes involves hybridization of labeled mRNA to an ordered array of oligonucleotides. Such a method allows the level of transcription of a plurality of these genes to be determined simultaneously to generate gene expression profiles or patterns. The gene expression profile derived from the sample obtained from the subject can be compared with the gene expression profile derived from the sample obtained from the disease-free subject to determine whether the genes are over-expressed in the sample from the subject relative to the genes in the sample obtained from the disease-free subject, and thereby determine whether the subject has or is at risk of developing schizophrenia, bipolar disorder and ADHD. The oligonucleotides utilized in this hybridization method typically are bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc. Any solid surface to which the oligonucleotides can be bound, either directly or indirectly, either covalently or non-covalently, can be used. A particularly preferred solid substrate is a high density array or DNA chip (see Example 2). These high density arrays contain a particular oligonucleotide probe in a pre-selected location on the array. Each pre-selected location can contain more than one molecule of the particular probe. Because the oligonucleotides are at specified locations on the substrate, the hybridization patterns and intensities (which together result in a unique expression profile or pattern) can be interpreted in terms of expression levels of particular genes. The oligonucleotide probes are preferably of sufficient length to specifically hybridize only to complementary transcripts of the above identified gene(s) of interest. As used herein, the term “oligonucleotide” refers to a single-stranded nucleic acid. Generally the oligonucleotides probes will be at least 16 to 20 nucleotides in length, although in some cases longer probes of at least 20 to 25 nucleotides will be desirable. The oligonucleotide probes can be labeled with one or more labeling moieties to permit detection of the hybridized probe/target polynucleotide complexes. Labeling moieties can include compositions that can be detected by spectroscopic, biochemical, photochemical, bioelectronic, immunochemical, electrical optical or chemical means. Examples of labeling moieties include, but are not limited to, radioisotopes, e.g., ³²P, ³³P, ³⁵S, chemiluminescent compounds, labeled binding proteins, heavy metal atoms, spectroscopic markers such as fluorescent markers and dyes, linked enzymes, mass spectrometry tags, and magnetic labels. Oligonucleotide probe arrays for expression monitoring can be prepared and used according to techniques which are well known to those skilled in the art as described, e.g., in Lockhart et al., Nature Biotechnology, Vol. 14, pp. 1675-1680 (1996); McGall et al., Proc. Natl. Acad. Sci. USA, Vol. 93, pp. 13555-13460 (1996); and U.S. Pat. No. 6,040,138.

Expression of the protein encoded by the gene(s) or a fragment of the protein, e.g., the catalytic domain, can be detected by a probe which is detectably labeled, or which can be subsequently labeled. Generally, the probe is an antibody which recognizes the expressed protein. As used herein, the term antibody includes, but is not limited to, polyclonal antibodies, monoclonal antibodies, humanized or chimeric antibodies and biologically functional antibody fragments, which are those fragments sufficient for binding of the antibody fragment to the protein or a fragment of the protein. For the production of antibodies to a protein encoded by one of the disclosed genes or to a fragment of the protein, various host animals may be immunized by injection with the polypeptide, or a portion thereof. Such host animals may include, but are not limited to, rabbits, mice and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above. Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature, Vol. 256, pp. 495-497 (1975); and U.S. Pat. No. 4,376,110), the human B-cell hybridoma technique (Kosbor et al., Immunology Today, Vol. 4, p. 72 (1983); Cole et al., Proc. Natl. Acad. Sci. USA, Vol. 80, pp. 2026-2030 (1983)), and the EBV-hybridoma technique (Cole et al., Monoclonal Antibodies and Cancer Therapy, Alan R. Uss, Inc., pp. 77-96 (1985)). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mAbs in vivo makes this the presently preferred method of production. In addition, techniques developed for the production of “chimeric antibodies” (Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region. Alternatively, techniques described for the production of single-chain antibodies (U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989)) can be adapted to produce differentially expressed gene-single chain antibodies. Single chain antibodies are formed by linking the heavy and light chain fragments of the Fv region via an amino acid bridge, resulting in a single-chain polypeptide. Most preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429. Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science, Vol. 246, pp. 1275-1281 (1989)) to allow rapid and easy identification of monoclonal Fab fragments with the desired specificity. The extent to which the known proteins are expressed in the sample is then determined by immunoassay methods which utilize the antibodies described above. Such immunoassay methods include, but are not limited to, dot blotting, western blotting, competitive and noncompetitive protein binding assays, enzyme-linked immunosorbant assays (ELISA), immunohistochemistry, fluorescence-activated cell sorting (FACS), and others commonly used and widely described in scientific and patent literature, and many employed commercially. Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested is brought into contact with the bound molecule and incubated for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay, in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the protein expressed by the gene of interest, or a fragment thereof. The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of secreted protein or fragment thereof. Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

In another aspect, kits are provided for detecting the level of expression of at least one gene identified in Tables 1, 2, and/or 3 in a biological sample, e.g., brain tissue (e.g. frontal pole and/or hypothalamus), with the proviso that if expression of only one gene is detected that the gene is not any one of the genes of Table 3. In some embodiments, if expression of only one gene is detected the gene is not any one of the genes of Table 3. For example, the kit can comprise a labeled compound or agent capable of detecting a protein encoded by, or mRNA corresponding to, at least one of the genes identified in Tables 1, 2, and/or 3 or fragment of the protein, means for determining the amount of protein encoded by or mRNA corresponding to the gene or fragment of the protein; and means for comparing the amount of protein encoded by or mRNA corresponding to the gene or fragment of the protein, obtained from the subject sample with a standard level of expression of the gene, e.g., from a disease-free subject. The compound or agent can be packaged in a suitable container. The kit can further comprise instructions for using the kit to detect protein encoded by or mRNA corresponding to the gene.

In another aspect, progression of schizophrenia, bipolar disorder and/or ADHD in a subject can be monitored by measuring a level of expression of mRNA corresponding to, or protein encoded by, at least one of the genes identified in Tables 1, 2, and/or 3 in a sample of brain tissue (e.g. frontal pole and/or hypothalamus) obtained in the subject over time, i.e., at various stages of the disorder, with the proviso that if expression of only one gene is detected that the gene is not any one of the genes of Table 3, measuring level of expression of mRNA corresponding to, or protein encoded by, at least one of the genes identified in Tables 5, 6 and/or 7 after an exposure to acute stress or e.g. dexamethasone. In some embodiments, if expression of only one gene is detected the gene is not any one of the genes of Table 3. An alteration, i.e. increase or decrease (depending on the disease associated up- or down-regulation of the Genes of the invention) in the level of expression of the mRNA or encoded protein corresponding to the gene(s) over time is indicative of the progression of schizophrenia, bipolar disorder and/or ADHD. The level of expression of mRNA and protein corresponding to the gene(s) can be detected by standard methods as described above. In a particularly useful embodiment, the level of mRNA expression of a plurality of the disclosed genes, at least two of the Genes of the invention, can be measured simultaneously in a subject at various stages of schizophrenia, bipolar disorder and/or ADHD to generate a transcriptional or expression profile of schizophrenia, bipolar disorder and/or ADHD over time. For example, mRNA transcripts corresponding to a plurality of these genes can be obtained from brain cells (e.g. from frontal pole and/or hypothalamus) of a subject at different times, and hybridized to a chip containing oligonucleotide probes which are complementary to the transcripts of the desired genes, to compare expression of a large number of genes at various stages of schizophrenia, bipolar disorder and/or ADHD.

In another aspect, an animal model-based assay based on the disclosed genes can be used to identify agents that can modulate the expression of one or more genes that are differentially expressed in subjects, e.g. human and/or animal models with schizophrenia, bipolar disorder and/or ADHD compared to subjects without schizophrenia, bipolar disorder and/or ADHD. Such agents are suitable for use in the treatment of schizophrenia, bipolar disorder and/or ADHD, and are useful in studies of the morphogenesis and progression of schizophrenia, bipolar disorder and/or ADHD. These methods generally involve comparing the expression level of one or more of the disclosed genes in a subject, e.g. in a sample of the frontal pole and/or hypothalamus that is contacted with a candidate agent to the expression level of the gene or genes in cells that are not contacted with the candidate agent. A suitable assay is shown in Example 4 and makes use of the animal model disclosed in this application. Thus, in some embodiments, the method comprises:
a) contacting the frontal pole and/or hypothalamus of a subject, e.g. animal model as defined herein with a candidate agent;
b) detecting a level of expression of at least one gene identified in Tables 1, 2 and/or 3 in the frontal pole and/or hypothalamus (=sample); and
c) comparing the level of expression of the gene in the sample in the presence of the candidate agent with a level of expression of the gene in the sample that is not contacted with the candidate agent, wherein a decreased or increased level of expression in the sample in the presence of the agent relative to the level of expression in the absence of the agent is indicative of an agent useful in the treatment of schizophrenia, bipolar disorder and/or ADHD.

In some embodiments, if expression of only one gene is detected the gene is not any one of the genes of Table 3. The level of expression of the gene is detected by measuring the level of mRNA corresponding to, or protein encoded by, the gene as described above. As used herein, the term “candidate agent” refers to any molecule that is capable of decreasing the level of mRNA corresponding to, or protein encoded by, at least one of the disclosed genes. The candidate agent can be natural or synthetic molecules such as proteins or fragments thereof, small molecule inhibitors, nucleic acid molecules, e.g., antisense nucleotides, ribozymes, double-stranded RNAs, organic and inorganic compounds and the like. Cell-based and/or cell-free assays can also be used to identify compounds which are capable of interacting with a protein encoded by one of the Genes of the invention or protein-binding partner to alter the activity of the protein or its binding partner. Cell-based and/or cell-free assays can also be used to identify compounds which modulate the interaction between the encoded protein and its binding partner such as a target peptide. In one embodiment, cell-based and/or cell-free assays for identifying such compounds comprise a cell expressing a protein encoded by any one of the Genes of the invention or a reaction mixture containing a protein encoded by one of the disclosed genes and a test compound or a library of test compounds in the presence or absence of the binding partner, e.g., a biologically inactive target peptide, or a small molecule. Accordingly, a cell-based and/or cell-free method for identifying agents useful in the treatment of schizophrenia, bipolar disorder and/or ADHD is provided which comprises contacting a protein or functional fragment thereof or the protein binding partner with a test compound or library of test compounds and detecting the formation of complexes. For detection purposes, the protein can be labeled with a specific marker and the test compound or library of test compounds labeled with a different marker. Interaction of a test compound with the protein or fragment thereof or the protein-binding partner can then be detected by measuring the level of the two labels after incubation and washing steps. The presence of the two labels is indicative of an interaction. Interaction between molecules can also be assessed by using real-time BIA (Biomolecular Interaction Analysis, Pharmacia Biosensor AB), which detects surface plasmon resonance, an optical phenomenon. Detection depends on changes in the mass concentration of mass macromolecules at the biospecific interface and does not require labeling of the molecules. In one useful embodiment, a library of test compounds can be immobilized on a sensor surface, e.g., a wall of a micro-flow cell. A solution containing the protein, functional fragment thereof, or the protein-binding partner is then continuously circulated over the sensor surface. An alteration in the resonance angle, as indicated on a signal recording, indicates the occurrence of an interaction. This technique is described in more detail in BIAtechnology Handbook by Pharmacia.

Another embodiment of a cell-free assay comprises:
a) combining a protein encoded by the at least one gene in Tables 1, 2 and/or 3, preferentaly at least one gene in Tables 1 and/or 2, the protein binding partner, and a test compound to form a reaction mixture; and
b) detecting interaction of the protein and the protein binding partner in the presence and absence of the test compounds.

A considerable change (potentiation or inhibition) in the interaction of the protein and binding partner in the presence of the test compound compared to the interaction in the absence of the test compound indicates a potential agonist (mimetic or potentiator) or antagonist (inhibitor) of the proteins activity for the test compound. The components of the assay can be combined simultaneously or the protein can be contacted with the test compound for a period of time, followed by the addition of the binding partner to the reaction mixture. The efficacy of the compound can be assessed by using various concentrations of the compound to generate dose response curves. A control assay can also be performed by quantitating the formation of the complex between the protein and its binding partner in the absence of the test compound. Formation of a complex between the protein and its binding partner can be detected by using detectably labeled proteins such as radiolabeled, fluorescently labeled, or enzymatically labeled protein or its binding partner, by immunoassay or by chromatographic detection. In preferred embodiments, the protein or its binding partner can be immobilized to facilitate separation of complexes from uncomplexed forms of the protein and its binding partner and automation of the assay. Complexation of the protein to its binding partner can be achieved in any type of vessel, e.g., microtitre plates, micro-centrifuge tubes and test tubes. In particularly preferred embodiments, the protein can be fused to another protein, e.g., glutathione-S-transferase to form a fusion protein which can be adsorbed onto a matrix, e.g., glutathione sepharose beads (Sigma Chemical. St. Louis, Mo.), which are then combined with the labeled protein partner, e.g., labeled with ³⁵S, and test compound and incubated under conditions sufficient to formation of complexes. Subsequently, the beads are washed to remove unbound label, and the matrix is immobilized and the radiolabel is determined. The aforementioned cell-free assays are particularly useful with proteins encoded by the Genes of the invention. Another method for immobilizing proteins on matrices involves utilizing biotin and streptavidin. For example, the protein can be biotinylated using biotin NHS(N-hydroxy-succinimide), using well known techniques and immobilized in the well of streptavidin-coated plates. Cell-free assays can also be used to identify agents which are capable of interacting with a protein encoded by the at least one gene and modulate the activity of the protein encoded by the gene. In one embodiment, the protein is incubated with a test compound and the catalytic activity of the protein is determined. In another embodiment, the binding affinity of the protein to a target molecule can be determined by methods known in the art.

The present invention also provides for both prophylactic and therapeutic methods of treating a subject having, or at risk of having, schizophrenia, bipolar disorder and/or ADHD. Subjects at risk for such disorders can be identified by a prognostic assay, e.g., as described above. Administration of a prophylactic agent can occur prior to the manifestation of symptoms characteristic of schizophrenia, bipolar disorder and/or ADHD, such that development of schizophrenia, bipolar disorder and/or ADHD is prevented or delayed in its progression. Examples of suitable therapeutic agents include, but are not limited to, antisense nucleotides, ribozymes, double-stranded RNAs and antagonists.

Hence, the present invention relates to a method of treating and preventing schizophrenia, bipolar disorder, and/or ADHD in patient in need thereof, the method comprising administering to said patient an effective amount of an agent that can induce a decrease in the expression of at least one gene with accession number X57514, AI639165, AF064868, AI145494, AA946532, U57500, L17127, L24776, AB020504, U75927, AF096269, AA892801, U49099, AA859597, AA875427, AF028784, L22760, AI014135, AI104513, AA894148, AI073204 and/or D38468; and/or induce an increase of at least one gene with accession number U20643, H31232, S61973, X85184, J04063, S81353, M33025, AA858621, M74494, AA866358, M64986, AI227715, AA89392, AA859633, AA891969, AI145367, AA893711, Y09000, AA891940, D26564, AI101103, M28648, AF014009, M13100, M59980, M18331, M36418, X57764, U62897, S71570, M16112, AI008131, X12744, AI230260, U48245, H31692, D89863, X06564, M91234, U31554AA875659, AA799421, AI014091, AB012234, U09793, AA800513, AI639381, AA799515, X83546, AI013194, AB008538, X52817, AA893065, H31588, AA859832, AI008074, AA851749, AA894321, AI227608, E13644, AA925762, M72422, M17526, U45479, U86635, AA894089, and/or AA891069; and/or for genes AB016160 and/or U77931 an increase with respect to frontal pole expression and a decrease to the hypothalamus expression; and/or for the gene U66707 a decrease with respect to the frontal pole and an increase with respect to the hypothalamus, with the proviso that if expression of only one gene is altered that the gene is not any gene of Table 3. In one embodiment of said method, the agent comprises an isolated nucleic acid molecule comprising an antisense nucleotide sequence derived from at least one gene with accession number X57514, U66707, AI639165, AF064868, U60578cds, AI145494, AA946532, AB016160, U57500, L17127, M28648, L24776, AB020504, U77931, U75927, AF096269, M892801, U49099, AA859597, AA875427, AF028784, L22760, AI014135, AI104513, AA894148, AI073204 or D38468. In a further embodiment of said method, the antisense nucleotide sequences are derived from at least two genes with accession number X57514, U66707, AI639165, AF064868, U60578cds, AI145494, AA946532, AB016160, U57500, L17127, M28648, L24776, AB020504, U77931, U75927, AF096269, AA892801, U49099, AA859597, AA875427, AF028784, L22760, AI014135, AI104513, AA894148, AI073204 or D38468. In another embodiment of said method, the at least one gene is selected from the group consisting of genes with accessions number U20643, H31232, S61973, AB016160, X85184, J04063, S81353, M33025, X57514, AA858621, M74494, M13100, M59980; AA946532, M18331, M36418, X57764, U62897, U57500, L17127, S71570, M16112, AI008131, M28648, X12744, AI230260, U86635, D38468. In one preferred embodiment of said method, the agent is an antagonist or agonist that inhibits a protein encoded by at least one gene with accession number X57514, U66707, AI639165, AF064868, U60578cds, AI145494, AA946532, AB016160, U57500, L17127, M28648, L24776, AB020504, U77931, U75927, AF096269, AA892801, U49099, AA859597, AA875427, AF028784, L22760, AI014135, AI104513, AA894148, AI073204 or D38468. In another preferred embodiment of said method, the agent is an antagonist or agonist that activates a protein encoded by at least one gene with accession number U20643, H31232, S61973, AB016160, X85184, J04063, S81353, M33025, AA858621, M74494, AA866358, M64986, AI227715, U77931, AA89392, AA859633, AA891969, AI145367, AA893711, Y09000, AA891940, D26564, AI101103, M28648, AF014009, M13100, M59980, M18331, M36418, X57764, U62897, S71570, M16112, AI008131, X12744, AI230260, U48245, U66707, H31692, D89863, X06564, M91234, U31554AA875659, AA799421, AI014091, AB012234, U09793, AA800513, AI639381, AA799515, X83546, AI013194, AB008538, X52817, AA893065, H31588, AA859832, AI008074, AA851749, AA894321, AI227608, E13644, AA925762, M72422, M17526, U45479, U86635, AA894089 or AA891069.

As used herein, the term “antisense” refers to nucleotide sequences that are complementary to a portion of an RNA expression product of at least one of the disclosed genes. “Complementary” nucleotide sequences refer to nucleotide sequences that are capable of base-pairing according to the standard Watson-Crick complementarity rules. That is, purines will base-pair with pyrimidine to form combinations of guanine:cytosine and adenine:thymine in the case of DNA, or adenine:uracil in the case of RNA. Other less common bases, e.g., inosine, 5-methylcytosine, 6-methyladenine, hypoxanthine and others may be included in the hybridizing sequences and will not interfere with pairing.

When introduced into a host cell, antisense nucleotide sequences specifically hybridize with the cellular mRNA and/or genomic DNA corresponding to the gene(s) so as to inhibit expression of the encoded protein, e.g., by inhibiting transcription and/or translation within the cell.

The isolated nucleic acid molecule comprising the antisense nucleotide sequence can be delivered, e.g., as an expression vector, which when transcribed in the cell, produces RNA which is complementary to at least a unique portion of the encoded mRNA of the gene(s). Alternatively, the isolated nucleic acid molecule comprising the antisense nucleotide sequence is an oligonucleotide probe which is prepared ex vivo and, which, when introduced into the cell, results in inhibiting expression of the encoded protein by hybridizing with the mRNA and/or genomic sequences of the gene(s).

Preferably, the oligonucleotide contains artificial internucleotide linkages which render the antisense molecule resistant to exonucleases and endonucleases, and thus are stable in the cell. Examples of modified nucleic acid molecules for use as antisense nucleotide sequences are phosphoramidate, phosporothioate and methylphosphonate analogs of DNA as described, e.g., in U.S. Pat. Nos. 5,176,996; 5,264,564; and 5,256,775. General approaches to preparing oligomers useful in antisense therapy are described, e.g., in Van der Krol, BioTechniques, Vol. 6, pp. 958-976 (1988); and Stein et al., Cancer Res., Vol. 48, pp. 2659-2668 (1988).

Typical antisense approaches, involve the preparation of oligonucleotides, either DNA or RNA, that are complementary to the encoded mRNA of the gene. The antisense oligonucleotides will hybridize to the encoded mRNA of the gene and prevent translation. The capacity of the antisense nucleotide sequence to hybridize with the desired gene will depend on the degree of complementarity and the length of the antisense nucleotide sequence. Typically, as the length of the hybridizing nucleic acid increases, the more base mismatches with an RNA it may contain and still form a stable duplex or triplex. One skilled in the art can determine a tolerable degree of mismatch by use of conventional procedures to determine the melting point of the hybridized complexes.

Antisense oligonucleotides are preferably designed to be complementary to the 5′ end of the mRNA, e.g., the 5′ untranslated sequence up to and including the regions complementary to the mRNA initiation site, i.e., AUG. However, oligonucleotide sequences that are complementary to the 3′ untranslated sequence of mRNA have also been shown to be effective at inhibiting translation of mRNAs as described, e.g., in Wagner, Nature, Vol. 372, pp. 333 (1994). While antisense oligonucleotides can be designed to be complementary to the mRNA coding regions, such oligonucleotides are less efficient inhibitors of translation.

Regardless of the mRNA region to which they hybridize, antisense oligonucleotides are generally from about 15 to about 25 nucleotides in length.

The antisense nucleotide can also comprise at least one modified base moiety, e.g., 3-methylcytosine, 5-methylcytosine, 7-methylguanine, 5-fluorouracil, 5-bromouracil, and may also comprise at least one modified sugar moiety, e.g., arabinose, hexose, 2-fluorarabinose, and xylulose.

In another embodiment, the antisense nucleotide sequence is an alpha-anomeric nucleotide sequence. An alpha-anomeric nucleotide sequence forms specific double stranded hybrids with complementary RNA, in which, contrary to the usual beta-units, the strands run parallel to each other as described e.g., in Gautier et al., Nucl. Acids. Res., Vol. 15, pp. 6625-6641 (1987).

Antisense nucleotides can be delivered to cells which express the described genes in vivo by various techniques, e.g., injection directly into the relevant brain tissue site, entrapping the antisense nucleotide in a liposome, by administering modified antisense nucleotides which are targeted to the relevant brain cells by linking the antisense nucleotides to peptides or antibodies that specifically bind receptors or antigens expressed on the cell surface.

However, with the above-mentioned delivery methods, it may be difficult to attain intracellular concentrations sufficient to inhibit translation of endogenous mRNA. Accordingly, in a preferred embodiment, the nucleic acid comprising an antisense nucleotide sequence is placed under the transcriptional control of a promoter, i.e., a DNA sequence which is required to initiate transcription of the specific genes, to form an expression construct. The use of such a construct to transfect cells results in the transcription of sufficient amounts of single stranded RNAs to hybridize with the endogenous mRNAs of the described genes, thereby inhibiting translation of the encoded mRNA of the gene. For example, a vector can be introduced in vivo such that it is taken up by a cell and directs the transcription of the antisense nucleotide sequence. Such vectors can be constructed by standard recombinant technology methods. Typical expression vectors include bacterial plasmids or phage, such as those of the pUC or Bluescript.™ plasmid series, or viral vectors such as adenovirus, adeno-associated virus, herpes virus, vaccinia virus and retrovirus adapted for use in eukaryotic cells. Expression of the antisense nucleotide sequence can be achieved by any promoter known in the art to act in mammalian cells. Examples of such promoters include, but are not limited to, the promoter contained in the 3′ long terminal repeat of Rous sarcoma virus as described, e.g., in Yamamoto et al., Cell, Vol. 22, pp. 787-797 (1980); the herpes thymidine kinase promoter as described, e.g., In Wagner et al., Proc. Natl. Acad. Sci. USA, Vol. 78, pp. 1441-1445 (1981); the SV40 early promoter region as described, e.g., in Bernoist and Chambon, Nature, Vol. 290, pp. 304-310 (1981); and the regulatory sequences of the metallothionein gene as described, e.g., in Brinster et al., Nature, Vol. 296, pp. 39-42 (1982).

Ribozymes are RNA molecules that specifically cleave other single-stranded RNA in a manner similar to DNA restriction endonucleases. By modifying the nucleotide sequences encoding the RNAs, ribozymes can be synthesized to recognize specific nucleotide sequences in a molecule and cleave it as described, e.g., in Cech, J. Amer. Med. Assn., Vol. 260, p. 3030 (1988). Accordingly, only mRNAs with specific sequences are cleaved and inactivated.

Two basic types of ribozymes include the “hammerhead”-type as described for example in Rossie et al., Pharmac. Ther., Vol. 50, pp. 245-254 (1991); and the hairpin ribozyme as described, e.g., in Hampel et al., Nucl. Acids Res., Vol. 18, pp. 299-304 (1999) and U.S. Pat. No. 5,254,678. Intracellular expression of hammerhead and hairpin ribozymes targeted to mRNA corresponding to at least one of the disclosed genes can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozyme sequences can be modified in essentially the same manner as described for antisense nucleotides, e.g., the ribozyme sequence can comprise a modified base moiety.

Double-stranded RNA, i.e., sense-antisense RNA, corresponding to at least one of the disclosed genes, can also be utilized to interfere with expression of at least one of the disclosed genes. Interference with the function and expression of endogenous genes by double-stranded RNA has been shown in various organisms such as C. elegans as described, e.g., in Fire et al., Nature, Vol. 391, pp. 806-811 (1998); drosophilia as described, e.g., in Kennerdell et al., Cell, Vol. 95, No. 7, pp. 1017-1026 (1998); and mouse embryos as described, e.g., in Wianni et al., Nat. Cell Biol., Vol. 2, No. 2, pp. 70-75 (2000). Such double-stranded RNA can be synthesized by in vitro transcription of single-stranded RNA read from both directions of a template and in vitro annealing of sense and antisense RNA strands. Double-stranded RNA can also be synthesized from a cDNA vector construct in which the gene of interest is cloned in opposing orientations separated by an inverted repeat. Following cell transfection, the RNA is transcribed and the complementary strands reanneal. Double-stranded RNA corresponding to at least one of the disclosed genes could be introduced into a relevant brain cell by cell transfection of a construct such as that described above.

The term “antagonist” refers to a molecule which, when bound to the protein encoded by the gene, inhibits its activity. Antagonists can include, but are not limited to, peptides, proteins, carbohydrates, and small molecules.

In a particularly useful embodiment, the antagonist is an antibody specific for the cell-surface protein expressed by the at least one Gene of the invention. Antibodies useful as therapeutics encompass the antibodies as described above. The antibody alone may act as an effector of therapy or it may recruit other cells to actually effect cell killing. The antibody may also be conjugated to a reagent such as a chemotherapeutic, radionuclide, ricin A chain, cholera toxin, pertussis toxin, etc., and serve as a target agent. Alternatively, the effector may be a lymphocyte carrying a surface molecule that interacts, either directly or indirectly, with a tumor target. Various effector cells include cytotoxic T cells and NK cells.

Examples of the antibody-therapeutic agent conjugates which can be used in therapy include, but are not limited to: 1) antibodies coupled to radionuclides, such as ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re, and as described, e.g., in Goldenberg et al., Cancer Res., Vol. 41, pp. 4354-4360 (1981); Carrasquillo et al., Cancer Treat. Rep., Vol. 68, pp. 317-328 (1984); Zalcberg et al., J. Natl. Cancer Inst., Vol. 72, pp. 697-704 (1984); Jones et al., Int. J. Cancer, Vol. 35, pp. 715-720 (1985); Lange et al., Surgery, Vol. 98, pp. 143-150 (1985); Kaltovich et al., J. Nucl. Med., Vol. 27, p. 897 (1986); Order et al., Int. J. Radiother. Oncol. Biol. Phys., Vol. 8, pp. 259-261 (1982); Courtenay-Luck et al., Lancet, Vol. 1, pp. 1441-1443 (1984); and Ettinger et al., Cancer Treat. Rep., Vol. 66, pp. 289-297 (1982); (2) antibodies coupled to drugs or biological response modifiers such as methotrexate, adriamycin, and lymphokines such as interferon as described, for, e.g., in Chabner et al., Cancer, Principles and Practice of Oncology, Philadelphia, Pa., J. B. Lippincott Co. Vol. 1, pp. 290-328 (1985); Oldham et al., Cancer, Principles and Practice of Oncology, Philadelphia, Pa., J. B. Lippincott Co., Vol. 2, pp. 2223-2245 (1985); Deguchi et al., Cancer Res., Vol. 46, pp. 3751-3755 (1986); Deguchi et al., Fed. Proc., Vol. 44, p. 1684 (1985); Embleton et al., Br. J. Cancer, Vol. 49, pp. 559-565 (1984); and Pimm et al., Cancer Immunol. Immunother., Vol. 12, pp. 125-134 (1982); (3) antibodies coupled to toxins, as described, for example, in Uhr et al., Monoclonal Antibodies and Cancer, Academic Press, Inc., pp. 85-98 (1983); Vitetta et al., Biotechnology and Bio. Frontiers, Ed. P. H. Abelson, pp. 73-85 (1984); and Vitetta et al., Science, Vol. 219, pp. 644-650 (1983); (4) heterofunctional antibodies, for example, antibodies coupled or combined with another antibody so that the complex binds both to the carcinoma and effector cells, e.g., killer cells such as T cells, as described, for example, in Perez et al., J. Exper. Med., Vol. 163, pp. 166-178 (1986); and Lau et al., Proc. Natl. Acad. Sci. USA, Vol. 82, pp. 8648-8652 (1985); and (5) native, i.e., non-conjugated or non-complexed antibodies, as described in, for example, Herlyn et al., Proc. Natl. Acad. Sci. USA, Vol. 79, pp. 4761-4765 (1982); Schulz et al., Proc. Natl. Acad. Sci. USA, Vol. 80, pp. 5407-5411 (1983); Capone et al., Proc. Natl. Acad. Sci. USA, Vol. 80, pp. 7328-7332 (1983); Sears et al., Cancer Res., Vol. 45, pp. 5910-5913 (1985); Nepom et al., Proc. Natl. Acad. Sci. USA, Vol. 81, pp. 2864-2867 (1984); Koprowski et al., Proc. Natl. Acad. Sci. USA, Vol. 81, pp. 216-219 (1984); and Houghton et al., Proc. Natl. Acad. Sci. USA, Vol. 82, pp. 1242-1246 (1985).

Methods for coupling an antibody or fragment thereof to a therapeutic agent as described above are well known in the art and are described, e.g., in the methods provided in the references above. In yet another embodiment, the antagonist useful as a therapeutic for treating schizophrenia, bipolar disorder of ADHD can be an inhibitor of a protein encoded by one of the disclosed genes. For example, the activity of the membrane-bound serine protease hepsin can be inhibited by utilizing specific serine protease inhibitors. Such serine-protease inhibitors are well known in the art as described, e.g., in Leung et al., “Protease Inhibitors: Current Status and Future Prospects”, J. Med. Chem., Vol. 43, pp. 305-341 (2000).

In the case of treatment with an antisense nucleotide, the method comprises administering a therapeutically effective amount of an isolated nucleic acid molecule comprising an antisense nucleotide sequence derived from at least one gene identified in Tables 1, 2, or 3 with the proviso that if expression of only one gene is inhibited that the gene is not any one of Table 3, wherein the antisense nucleotide has the ability to decrease the transcription/translation of the at least one gene. In some embodiments, if expression of only one gene is inhibited the gene is not any one of Table 3. The term “isolated” nucleic acid molecule means that the nucleic acid molecule is removed from its original environment (e.g., the natural environment if it is naturally occurring). For example, a naturally occurring nucleic acid molecule is not isolated, but the same nucleic acid molecule, separated from some or all of the co-existing materials in the natural system, is isolated, even if subsequently reintroduced into the natural system. Such nucleic acid molecules could be part of a vector or part of a composition and still be isolated, in that such vector or composition is not part of its natural environment.

With respect to treatment with a ribozyme or double-stranded RNA molecule, the method comprises administering a therapeutically effective amount of a nucleotide sequence encoding a ribozyme, or a double-stranded RNA molecule, wherein the nucleotide sequence encoding the ribozyme/double-stranded RNA molecule has the ability to decrease the transcription/translation of at least one gene identified in Tables 1, 2, or 3, with the proviso that if expression of only one gene is inhibited that the gene is not any gene of Table 3.

In the case of treatment with an antagonist or agonist, the method comprises administering to a subject a therapeutically effective amount of an antagonist that inhibits a protein encoded by at least one gene identified in Tables 1, 2, or 3, with the proviso that if expression of only one gene is inhibited that the gene is not any gene of Table 3.

A “therapeutically effective amount” of an isolated nucleic acid molecule comprising an antisense nucleotide, nucleotide sequence encoding a ribozyme, double-stranded RNA, or antagonist, refers to a sufficient amount of one of these therapeutic agents to treat schizophrenia, bipolar disorder, ADHD. The determination of a therapeutically effective amount is well within the capability of those skilled in the art. For any therapeutic, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Antisense nucleotides, ribozymes, double-stranded RNAs, and antagonists which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range, depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for antagonists.

For therapeutic applications, the antisense nucleotides, nucleotide sequences encoding ribozymes, double-stranded RNAs (whether entrapped in a liposome or contained in a viral vector) and antibodies are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions may be administered by any number of routes, including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated from aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0. 1-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of the antisense nucleotide or antagonist, such labeling would include amount, frequency, and method of administration. Those skilled in the art will employ different formulations for antisense nucleotides than for antagonists, e.g., antibodies or inhibitors. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

In another aspect, the treatment of a subject with a therapeutic agent, such as those described above, can be monitored by detecting the level of expression of mRNA or protein encoded by at least one of the disclosed genes identified in Tables 1 or 2, or the activity of the protein encoded by the at least one gene. These measurements will indicate whether the treatment is effective or whether it should be adjusted or optimized. Accordingly, one or more of the genes described herein can be used as a marker for the efficacy of a drug during clinical trials.

In a particularly useful embodiment, a method for monitoring the efficacy of a treatment of a subject having schizophrenia, bipolar disorder and/or ADHD, or at risk of. or having schizophrenia, bipolar disorder and/or ADHD with an agent (e.g., an antagonist, protein, nucleic acid, small molecule, or other therapeutic agent or candidate agent identified by the screening assays described herein) is provided comprising:
a) obtaining a pre-administration sample from a subject prior to administration of the agent,
b) detecting the level of expression of mRNA corresponding to, or protein encoded by the at least one gene, or activity of the protein encoded by the at least one gene identified in Tables 1 or 2 in the pre-administration sample;
c) obtaining one or more post-administration samples from the subject,
d) detecting the level of expression of mRNA corresponding to, or protein encoded by the at least one gene, or activity of the protein encoded by the at least one gene in the post-administration sample or samples,
e) comparing the level of expression of mRNA or protein encoded by the at least one gene, or activity of the protein encoded by the at least one gene in the pre-administration sample with the level of expression of mRNA or protein encoded by the at least one gene, or activity of the protein encoded by the at least one gene in the post-administration sample or samples, and
f) adjusting the administration of the agent accordingly.

In some embodiments, if expression corresponding to, or activity of protein encoded by, only one gene is detected the gene is not any gene of Table 3. In other embodiments, if expression corresponding to, or activity of protein encoded by, only one gene is detected the gene is not any gene of Table 3. For example, increased administration of the agent may be desirable to decrease the level of expression or activity of the at least one gene to lower levels than detected, i.e., to increase the effectiveness of the agent. Alternatively, decreased administration of the agent may be desirable to increase expression or activity of the at least one gene to higher levels than detected, i.e., to decrease the effectiveness of the agent.

In another aspect, a viral vector is provided which comprises a promoter and/or an enhancer or other regulatory element of a gene selected from the group consisting of at least one of the genes identified in Tables 1, 2, or 3 operably linked to the coding region of a gene that is essential for replication of the vector, wherein the vector is adapted to replicate upon transfection into a diseased cell. The promoter sequences can be discerned by searching the publicly available databases for BAC clones that cover the entire gene; thereafter, the cDNA for the gene can be compared to the genomic sequence. This will generally reveal the intron-exon boundaries and the start site of the gene. Once these are established, the promoter sequences can be inferred. Such vectors are able to selectively replicate in a brain cell, e.g. frontal lobe and/or hypothalamus, of a patient having schizophrenia, bipolar disorder and/or ADHD, but not in a non-diseased cell. The replication is conditional upon the presence in a diseased cell, and not in a non-diseased (=standard expression profile) cell, of positive transcription factors that activate the promoter of the disclosed genes. It can also occur by the absence of transcription inhibiting factors that normally occur in a non-diseased cell and prevent transcription as a result of the promoter. Accordingly, when transcription occurs, it proceeds into the gene essential for replication, such that in the diseased cell, but not in non-diseased cell, replication of the vector and its attendant functions occur. With this vector, a diseased cell, e.g., a brain cell, e.g. hypothalamus and/or frontal lobe of a patient having schizophrenia, bipolar disorder and/or ADHD, can be selectively treated, with minimal systemic toxicity.

In one embodiment, the viral vector is an adenoviral vector, which includes a coding region of a gene essential for replication of the vector, wherein the coding region is selected from the group consisting of E1a, E1b, E2 and E4 coding regions. The term “gene essential for replication” refers to a nucleic acid sequence whose transcription is required for the vector to replicate in the target cell. Preferably, the gene essential for replication is selected from the group consisting of the E1A and E1b coding sequences. Particularly preferred is the adenoviral E1A gene as the gene essential for replication. Methods for making such vectors are well know to the person of ordinary skill in the art as described, e.g., in Sambrook et al., in Molecular Cloning: A Laboratory Manual, Cold Spring Harbor, N.Y., 1989.

In a further embodiment, the invention provides nucleic acid constructs in which a heterologous gene product is expressed under the control of a promoter and/or an enhancer or other regulatory element of a gene selected from the group consisting of at least one of the genes identified in Tables 1, 2, 3, 5, 6 or 7. Such heterologous gene products are expressed when the construct is present in diseased cells, but not in normal, non-diseased cells. The heterologous gene product provides, in some embodiments, for the inhibition, prevention, or destruction of the growth of the diseased cell. The gene product can be RNA, e.g., antisense RNA or ribozyme, or proteins such as a cytokine, e.g., interleukin, interferon, or toxins such as diphtheria toxin, pseudomonas toxin, etc. The heterologous gene product can also be a negative selective marker such as cytosine deaminase. U.S. Pat. No. 6,057,299, for example, describes the construction and use of nucleic acid constructs in which heterologous genes are placed under the control of a PSA enhancer. The nucleic acid constructs can be introduced into target cells by methods known to those of skill in the art. For example, one can incorporate the constructs into an appropriate vector such as those described above.

The vector of the present invention can be transfected into a helper cell line for viral replication and to generate infectious viral particles. Alternatively, transfection of the vector or other nucleic acid into a nerve cell can take place by electroporation, calcium phosphate precipitation, microinjection, or through liposomes, including proteoliposomes.

In a further embodiment, the invention provides a novel heuristic animal model for schizophrenia, bipolar disorder and/or ADHD as shown in example 1.

The following examples are included to demonstrate preferred embodiments of the invention.

### EXAMPLE 1

### Animal Model

A) The repeated variable prenatal stress paradigm: Timed pregnant Sprague Dawley rats (Charles River Laboratories, USA) are subjected to a variable repeated stress during the last week of the gestation, starting on embryonic day (E) 14 and continuing until the natural delivery of the pups at E22. The stress paradigm consists of a 1 h restraint in the cylindrical restrainers (×3/day), exposure to a cold environment (+4° C., 6 h), over night food deprivation, swim stress (×3/day) and reversal of the light-dark cycle. All of the mothers are exposed to the same stressors but in a randomized manner to prevent accomodation and, the same stresses are applied at the same time of the day. During the prenatal stress period, all stresses are performed at least twice. Control dams remain in the animal room and are not exposed to any unusual procedures. Following the delivery, the dam and her pups are left undisturbed in their cages until weaning on the postnatal day (P) 21, when the male and the female offspring are separated and housed with 1-2 littermates per cage with free access to food and water.

B) The acute stress procedure: On the morning of P56 (at 0900 h), after an acclimation period of 5-7 d in the laboratory environment to re-establish homeostasis and basal levels of gene expression after a considerable release of corticosterone induced by moving the animals from animal room to the laboratory (The laboratory environment is soundproof, and has its own ventilation system. An acclimation period of min. 48 h was required to re-establish homeostasis and basal levels of gene expression J. Koenig, unpublished observation.), the prenatally stressed offspring and the prenatally non-stresssed controls are placed in cylindrical plastic restrainers for 30 min. At the end of the acute restraint session, the animals were either sacrificed by decapitation or returned to their cages to be sacrificed at 120 min or 24 h after removal from the restraint device. Rats designated to the baseline group to establish the basal corticosterone levels and gene expression are sacrificed without exposure to acute stress throughout the course of the experiment to control for circadian differences between the animals.

C) Behavioral assessment: Behavioral assessments consisting of basal and psychomotor stimulant-induced locomotor activity, prepulse inhibition (PPI; a measure of sensorimotor gating; Loss of normal PPI has been thought to be a measure of sensorymotor gating and has been shown to be disrupted in schizophrenia; Braff and Geyer, Arch Gen Psychiatry. 47:181-188 (1987); Geyer and Braff, Schizophr Bull. 13:643-668 (1990)), N40 (auditory evoked potential, measure of sensory gating in rats; Bickford-Wimer et al., Biol Psychiatry. 27:183-192 (1990)) and acute stress reactivity are done on the male offspring on postnatal days 35 and 56. Control animals are obtained from dams not exposed to stress during gestation. Prenatal stress exposure induce a heightened locomotor response to both amphetamine and phencyclidine only on post-natal day 56. Moreover, sensorimotor gating assayed by both PPI and N40 are disrupted in animals exposed to prenatal stress. Deficits in glucocorticoid feedback are noted in animals exposed to prenatal stress on post-natal day 56. Plasma corticosterone levels are measured in prenatally stressed and the non-stressed controls at baseline and, 30 min., 2 h and 24 h after acute stress exposure. The prenatally stressed show a prolonged elevation of corticosterone after the acute stress.

The animals used in this study are maintained in fascilities fully accredited by the American Association for the Accreditation of laboratory Animal care (AAALAC) and the studies are conducted in accordance with the Guide for Care and Use of Laboratory Animals provided by the NIH.

### EXAMPLE 2

### Identification of Potential Diagnostic Markers and Therapeutic Targets

A) Probe preparation and microarray hybridization: The brains are removed and frontal pole and hypothalamus are quickly dissected, placed in finely powdered dry ice till the samples are frozen and then wrapped in aluminium foil. For dissection of the frontal pole, the brain is placed on the dorsal side and a clean razor blade is used to make a frontal cut beginning at the rostral pole of the olfactory tubercle and proceeding through the dorsal cortex. For hypothalamus, the brain is placed on the dorsal side and a clean razor blade is used to make a vertical cut at the rostral tip of the optic chiasm and behind the mammillary bodies. Cuts on the lateral aspects are made in the hippocampal sulci. The cuts are defined by the Circle of Willis. Once this piece of tissue is isolated, it is cut at a thickness of 1.5 mm which contains all the hypothalamic nuclei and thus is without contamination by the thalamus or other brain region. The samples are stored at −80° C. until isolation of the total RNA.

Total RNA is isolated using peqGOLD RNAPure™ (PEQLAB Biotechnologie GmbH, Germany), total RNA is DNAse treated (10 U RNAse free DNAse 2 U/μl; Ambion and 1 U/μl of the final volume RNAse inhibitor SuperasIn; 20 U/μl; Ambion; USA) and repurified using RNeasy (Qiagen Inc., USA). Thereafter, the samples are labeled and hybridized individually on rat genome RG-U34A microarrays (Affymetrix, Santa Clara, Calif., USA) as previously described (Lockhart et al., Nat Biotechnol. 14:1675-1680 (1996); Affymetrix Genechip® Expression Analysis Technical Protocol (2001) Affymetrix, Inc., USA). Primary image analysis of the arrays is performed using the Genechip 3.2 software package (Affymetrix, Santa Clara, Calif., USA) and images are scaled to an average hybridization intensity (average difference value) of 150. For the frontal pole samples (n=48) the background, the percentage of present values, the AFFX-GAPDH3′/AFFX-GAPDH5′ probe expression ratio and the AFFX-GAPDH3′ probe variation are 62.5±9.45, 40.89±3.01, 6.86±7.36 and 23% respectively and, for the hypothalamus samples (n=35) 69.61±9.81, 44.78±2.54, 2.07±0.99 and 11%, which indicates that the gene chips are of good quality and the variation is relatively small, as compared to other studies performed using similar method and material.

B) Validation of the animal model: Hierarchical clustering of the experiment based on Pearson correlation around zero of the gene expression of all 8799 genes on the Affymetrix RGU34A microarray is performed for the frontal pole samples. Each timepoint (baseline (BL), 24 h, 30 minutes, 2 h) reflects the average expression of six individual samples in that group. The non-stressed controls and the prenatally stressed clustered separately. In both main treatment groups, the global gene expression at the 2 h timepoint after acute stress differs most from the BL situation. The gene expression returns close to the baseline 24 h after the acute stress in the prenatally non-stressed animals. However, in the prenatally stressed, the gene expression at 24 h after acute stress resembled more closely the 30 min or the 2 h situation than the BL situation thus reflecting a prolonged stress response at the global gene expression level in the prenatally stressed animals. This molecular pattern thus goes well and fits well with the behavior changes observed in the animals of example 1.

B) Microarray analysis: Microarray analysis is performed using GeneSpring® 4.2.1 (Silicon Genetics, Redwood City, Calif., USA) and Novartis Pharmacogenetics Gene Expression Analysis Tools (for description see U.S. Ser. No. 60/377,446). Three different normalization approached are followed by using raw data, the minimum change of gene expression and a statistical restriction exercise (see below) to arrive at a list of consistently differentially expressed genes that occurred in common in at least two separate comparisons based on different normalizations. The normalization approaches used are:

Approach 1) The expression value for each gene on a chip is divided by the mean of all expression values on that chip assuming that this is at least 10. The bottom 10% is used as a test for correct background subtraction. Thereafter, each gene is normalized to itself by dividing all measurements for that gene by the median of the gene's expression values over all the samples. Only genes having a raw data expression value ≧100 at least in one of the two conditions (non-stressed vs. stressed) are further analysed. Furthermore, genes showing a minimum fold difference of 1.5 in the expression between the non-stressed and the stressed animals with a statistical significance p≦0.005 (Wilcoxon test) are considered as differentially expressed genes, i.e. 29 genes in the frontal lobe and 82 genes in the hypothalamus fulfill this criteria.

Approach 2) The expression value for each gene on a chip is divided by the mean of all expression values on that chip. Thereafter, GAPDH 3′ expression on each chip is divided by the average of GAPDH 3′ expression across the whole sample set. Result of the GAPDH 3′/Avg GAPDH 3′ is then used to divide the expression values for other genes on that chip. The procedure brings the GAPDH 3′ expression value on every chip to the same value, and thus normalizes the global gene expression with respect to GAPDH 3′. Again, only genes having raw data expression values ≧100 at least in one of the two conditions (non-stressed vs. stressed) are further selected. Thereafter, genes showing a minimum fold difference of 1.5 in the expression between the non-stressed and the stressed animals with a statistical significance p≦0.005 (Wilcoxon test) are considered as differentially expressed, i.e. 42 genes in the frontal lobe and 139 genes in the hypothalamus fulfill this criteria.

Approach 3) The signal intensity on each chip has been scaled to 150 (arbitrary intensity based on experience), thus no normalization is applied to this set of data. Genes are selected based on logarithmic average expression difference between the study groups. Genes showing a minimum fold difference of 1.5 in the expression between the non-stressed and the stressed with a statistical significance p≦0.005 (Wilcoxon test) are considered as differentially expressed, i.e. 75 genes in the frontal lobe and 100 genes in the hypothalamus fulfill this criteria.

Thus, only genes that are identified as being differentially expressed in more than one approach shown above are included on a list of constantly differentially expressed genes, i.e. 31 genes in the frontal pole (see Table 1) and 66 genes (see Table 2) in the hypothalamus fulfill this final criteria.

Arbitrary expression levels of Genes of the invention are shown to demonstrate the differential levels of expression of said genes as shown in Table 1 and 2 below:

TABLE 1
Transcript Levels in the frontal pole of prenatally stressed rats compared
to non stressed rats

| Accession no. | Gene name | Up/down regulated |
|---|---|---|
| U20643 | Aldolase A | down |
| H31232 | Cox IV-2 | down |
| S61973 | NMDA receptor glutamate-binding subunit (grina) | down |
| AB016160 | GABA-B receptor 1c | down |
| X85184 | Ras-related GTPase, ragB | down |
| J04063 | CamKII gamma | down |
| S81353 | Sulfated glycoprotein-1; Prosaposin | down |
| M33025 | Parathymosin | down |
| X57514 | GABA(A) receptor gamma-1 subunit | up |
| AA858621 | CamKII inhibitor alpha | down |
| AA866358 | EST, similar to 2310042M24; by synteny CGI-31 | down |
| U66707 | Densin-180 | up |
| M64986 | Rat amphoterin/Hmgb1 (high motility group box 1) | down |
| AI227715 | Retinoblastoma-like 2 | down |
| U77931 | Ribin | down |
| AI639165 | cDNA; 94% similar to FLJ11753 | up |
| AA89392 | 4 Kruppel-like factor 13 | down |
| AA859633 | EST, similar to PD2 protein | down |
| AA891969 | Nuclear DNA-binding protein (C1D) | down |
| AI145367 | Cap2, adenylyl cylase-associated protein 2 | down |
| AF064868 | Begain | up |
| AA893711 | EST similar to HS44 mouse; Dnaj (Hsp40) | down |
| Y09000 | Dendrin | down |
| AA891940 | EST highly similar to RHOC (ras homolog 9) | down |
| D26564 | RATCDS37, similar to cdc37 | down |
| AI101103 | Vamp2 (synaptobrevin II) | down |
| M74494 | Na, K-ATPase alpha-1 subunit | down |
| AF014009 | Acidic calcium-independent phospholipase A2 | down |
| AI145494 | Synapsin 2 | up |
| (Notes: | | |
| Accession number can be used to identify the unique identity of each gene at NCBI - UniGene at http://www.ncbi.nlm.nih.gov/UniGene/; | | |
| “up/down regulation” indicates that the genes is differentially expressed in the frontal pole | | |
| # according to the criteria laid down in example 2, i.e. an at least 1.5 fold difference in the expression between the non-stressed and the stressed animals with a statistical significance of p ≦ 0.005 (Wilcoxon test); | | |

i.e. “up” indicates an up regulation whereas “down” indicates a down regulation)

TABLE 2
Transcript Levels in the hypothalamus of prenatally stressed rats
compared to non-stressed rats

| | | Up/down |
|---|---|---|
| Accession no. | Gene name | regulated |
| M13100 | Heme oxygenase-3 (HO-3) | down |
| M59980 | Voltage-gated K+ channel protein (RK5) | down |
| AA946532 | Abcd3 | up |
| M18331 | Protein kinase C epsilon | down |
| M36418 | Glutamate receptor (GluR-A, gria1, glur1) | down |
| AB016160 | GABAB receptor 1c | up |
| X57764 | ET-B endothelin receptor | down |
| U62897 | Carboxypeptidase D precursor (Cpd) | down |
| U57500 | Protein tyrosine phosphatase alpha | up |
| L17127 | psmb4; proteasome (prosome, macropain) | up |
| S71570 | CaMKII gamma-b | down |
| M16112 | CaMKII beta | down |
| AI008131 | S-adenosylmethionine decarboxylase | down |
| M28648 | Na, K-ATPase alpha-2 | up |
| X12744 | c-erb-A thyroid hormone receptor | down |
| AI230260 | casein kinase II beta | down |
| U48245 | PKC-binding protein Nel | down |
| U66707 | Densin-180 | down |
| H31692 | eif2C2 (Gerp95) | down |
| D89863 | M-Ras | down |
| X06564 | NCAM | down |
| M91234 | VL30 element | down |
| U31554 | Limbic system-associated membrane | down |
| | protein | |
| L24776 | Tropomyosin non-muscle isoform NM3 | up |
| AB020504 | PMF31 | up |
| U77931 | Ribin | up |
| U75927 | Cytochrome oxidase subunit VIIa | up |
| AF096269 | epsin 2 | up |
| AA892801 | Eef2 | up |
| U49099 | Golgi SNAP receptor complex 1 (Gosr1) | up |
| AA875659 | alpha internexin | down |
| AA859597 | ESTs, similar to Kcnk6 | up |
| AA875427 | Aes | up |
| AA799421 | ESTs, similar to PKC epsilon | down |
| AI014091 | Cited 2 | down |
| AB012234 | NF1-X1 | down |
| U09793 | p21 (c-Ki-ras); kras2 | down |
| AA800513 | ESTs; similar to AI481500 and K08H10 | down |
| AF028784 | GFAP alpha and delta | up |
| AI639381 | ESTs, similar to Ras suppressor 1 | down |
| AA799515 | ESTs, similar to WSB-2 | down |
| X83546 | Leucocyte common antigen-related (lar2) | down |
| AI013194 | elF-5 | down |
| AB008538 | alcam | down |
| X52817 | C1-13 gene product; reticulon 1 | down |
| AA893065 | ESTs, similar to NPEPPS | down |
| H31588 | ESTs, similar to 2210408F11 gene | down |
| AA859832 | ESTs, similar to AK004010 and | down |
| | AP001458.5 | |
| AI008074 | ESTs, similar to HSP90 beta | down |
| AA851749 | Sfrs10 | down |
| AA894321 | ESTs, similar to FENS1 | down |
| AI227608 | microtubule-associated protein tau | down |
| L22760 | GATA-GT1 (gata6) | up |
| AI014135 | beta-carotene 15 | up |
| AI104513 | cytochrome c oxidase subunit Va | up |
| AA894148 | ESTs, similar to ApoA-IV | up |
| AI073204 | Ywhae | up |
| E13644 | Neurodap-1 | down |
| AA925762 | Marcs | down |
| M72422 | Glutamic acid decarboxylase (GAD65) | down |
| M17526 | GTP-binding protein (G-alpha-0) | down |
| U45479 | Synaptojanin; synj1 | down |
| U86635 | Glutathione S-transferase M | down |
| D38468 | BIT | up |
| AA894089 | Neurodegeneration-associated protein 1 | down |
| AA891069 | ESTs, similar to SFRS protein kinase 2 | down |
| (Notes: | | |
| Accession number can be used to identify the unique identity of each gene at NCBI - UniGene at http://www.ncbi.nlm.nih.gov/UniGene/; | | |
| “up/down regulation” indicates that the genes is differentially expressed in the hypothalamus according to the criteria laid down in example 2, i.e. an at least 1.5 fold difference in the expression between the non-stressed | | |
| # and the stressed animals with a statistical significance of p ≦ 0.005 (Wilcoxon test); | | |

i.e. “up” indicates an up regulation whereas “down” indicates a down regulation)

TABLE 3
Genes identified in Table 1 and 2 which are known to be associated with
Schizophrenia, ADHD and/or bipolar disorder

| Accession no. | Gene name | Reference |
|---|---|---|
| AI101103 | Vamp2 (synaptobrevin II) | Sokolov et al., (2000) |
| | | Biol. Psych. 48: 184-196. |
| | | 196. |
| M72422 | Glutamic acid decarboxylase (GAD65) | Hakak et al., (2001) |
| | | Proc. Nat. Acad. Sci., |
| | | 98: 4746-4751. |
| M17526 | GTP-binding protein (G-alpha-0) | Tani et al., (2001) |
| | | Mol. Psychiatry 6: |
| | | 359. |
| AF014009 | acidic calcium-independent phospholipase | Ross et al. (1997) |
| | A2 (aiPLA2) | Arch. Gen. Psych. |
| | | 54: 487-494. Ross et |
| | | al (1999) Brain Res. |
| | | 821: 407-413. |
| U45479 | Synaptojanin; synj1 | Saito et al. (2001) Mol |
| | | Psychiatry 6: 387-395. |
| X06564 | NCAM | Barbeau et al.(1995) |
| | | Proc. Natl. Acad. Sci. |
| | | USA 92: 2785-2789 |
| AI145494 | Synapsin 2 | Mirnics et al., (2000) |
| | | Neuron 28: 53-67. |

### EXAMPLE 3

### Real-Time (RT) PCR: Confirmation of Differentially Regulated Genes

Nine genes from the 35 most constantly differentially expressed in the frontal pole are chosen based on their drugability and validation value (e.g. genes that are already known to be associated e.g. with schizophrenia (see Table 3)) for quantitation using a fluorescence-based real time PCR (Taqman, Applied Biosystems, Foster City, Calif.; Gibson et al., 1996; Heid et al., 1996). The following primers and probes (Microsynth, Balgach, Switzerland) are designed using ABI PrimerExpress software:

TABLE 4
The sequence of the probe pairs for RT-PCR:

| | | |
|---|---|---|
| | | SEQ |
| Primer Name | Sequence 5′-3′ | ID NO: |

| | | |
|---|---|---|
| AiPLA2- | CAGCTGCAGTTCCGTAGAAAGA | 1 |
| Forward(F) | | |
| AiPLA2- | GCGAGCGACCTACGCG | 2 |
| Reverse(R) | | |
| AiPLA2-Probe | TGTGGCGTGGTCACAGCCGAAG | 3 |
| (TQ) | | |
| Aldolase A-F | AGGAAGAGGCATCCATCAACC | 4 |
| Aldolase A-R | GAAAGTCAAGGCCCATGGC | 5 |
| Aldolase A-TQ | CAATGCTATCAACAAGTGTCCCCTGCTGA | 6 |
| Densin 180-F | GCCTTGACCACCCTGGAAA | 7 |
| Densin 180-R | GCTCCGCTGGAACTGATACAG | 8 |
| Densin 180-TQ | TAATCACGGCGTTTGCGCGGT | 9 |
| GabaB1c-F | TGGAGAGCTTTGGTCTTTTGC | 10 |
| GabaB1c-R | GGGTGAGTCACCGCCTACAG | 11 |
| GabaB1c-TQ | TGAGTAGTGATGTTCAGCGGAGGGCC | 12 |
| Grina-F | CAGCTCAGGTGGCATGGTG | 13 |
| Grina-R | GGAAGCACAGATGGCAATGTC | 14 |
| Grina-TQ | CTCAGACCCATGCCCCTGCCA | 15 |
| PGK1-F | GCCAACTCGGTTGTGCTTATG | 16 |
| PGK1-R | GTACTTGTCGGGCATGGGC | 17 |
| PGK1-TQ | CCACCTGGGCCGTCCTGATGGT | 18 |
| Ribin-F | CAGAGGCTGTTCACCTTGGAG | 19 |
| Ribin-R | AGGGTGTAAATCTCGCGCC | 20 |
| Ribin-TQ | TGCTGCGGATATGGGTACGGCC | 21 |
| Synapsin2-F | ACAGATGTCCAAGCCCCCA | 22 |
| Synapsin2-R | CGCCATGTCAGACCGTACAA | 23 |
| Synapsin2-TQ | ACATCTCAGAGCAAGCGTCGACCCAG | 24 |
| Vamp2-F | CTGCCTGATCTGCTGCCTC | 25 |
| Vamp2-R | CACCCCTCCTCAAAGAACCA | 26 |
| Vamp2-TQ | ACCAGGAGAACTGGAGGCTGACCACA | 27 |

All the Taqman probes are 5′FAM (6-carboxyfluorescein)- and 3′ TAMRA (6-carboxytetramethylrhodamine)-labeled. BLAST searches are performed to confirm primer and probe specificity. Baseline (n=12) and 2 h timepoint (n=11) samples are chosen as template. The amount of total RNA is determined using RiboGreen (Molecular Probes, Eugene, Oreg., USA) RNA quatitation method. 400 ng of the total RNA (15 ng/μl) is treated with DNAse I mix (RNAse free DNAse kit, Qiagen, USA). 5 ng of the DNAse-treated RNA is used to control for genomic DNA-contamination using the real time PCR and if negative, 250 ng of the RNA is reverse transcribed using random priming and Omniscript RT Kit (Qiagen, USA) as described by the manufacturer. 5 ng of the resulting cDNA is used as a template for the following PCR reaction for which the conditions are optimized so that primers could be used at 300 nM and the probes at 175 nM concentration with qPCR Mastermix (Eurogentec, Seraing, Belgium) in a 25 μl reaction. The thermal cycle conditions used are as follows: 2 min 50° C., 10 min 95° C., followed by 40 two-step cycles at 95° C. for 15 s and one min at 60° C. The relative standard curve method (User Bulletin 2, PE Applied Biosystems, 1997) is used to determine the amount of mRNA of the gene of interest relative to an endogenous control gene. Phosphoglycerate kinase I (pgk1) is chosen as an endogenous control gene because it shows the smallest intersample variation and response to acute stress in our microarray data set (data not shown). For each gene, the samples are run as triplicates or duplicates, and the reactions are repeated at least once. The reactions for the gene of interest and for the endogenous control are always performed on the same reaction plate.

### EXAMPLE 4

### Validation of the Differentially Expressed Genes in the Animal Model by Known Drugs to Schizophrenia, ADHD and Bipolar Disorder

Timely impregnated Sprague Dawley rats (Charles River Laboratories, USA) are subjected to a variable repeated stress during the last week of the gestation, starting on embryonic day (E) 14 and continuing until the natural delivery of the pups at E22. The stress paradigm consists of a 1 h restraint in the cylindrical restrainers (×3/day), exposure to a cold environment (+4° C., 6 h), over night food deprivation, swim stress (×3/day) and reversal of the light-dark cycle. All of the mothers are exposed to the same stressors but in a randomized manner to prevent accomodation and, the same stresses are applied at the same time of the day. During the prenatal stress period, all stresses are performed at least twice. Control dams remain in the animal room and are not exposed to any unusual procedures. Following the delivery, the dam and her pups are left undisturbed in their cages until weaning on the postnatal day (P) 21, when the male and the female offspring are separated and housed with 1-2 littermates per cage with free access to food and water.

On postnatal day (P56) the prenatally stressed and the non-stressed controls are divided into corresponding treatment groups and their baseline sensory motor gating of is examined using prepulse inhibition (PPI) paradigm. The treatment groups are as follows:

Acute Treatment:

1) Prenatally stressed: Haloperidol i.p. acute dosing regime.

2) Non-stressed controls: Haloperidol i.p., acute dosing regime.

3) Prenatally stressed: Clozapine i.p. acute dosing regime.

4) Non-stressed controls: Clozapine i.p. acute dosing regime.

5) Prenatally stressed: vehicle i.p. (acute dosing control).

6) Non-stressed controls: vehicle i.p. (acute dosing control).

7) Prenatally stressed, methylphenidate i.p. acute dosing regime.

8) Non-stressed controls, methylphenidate i.p. acute dosing regime.

Immediately after acute dosing, the animals are tested for their PPI and sacrificed thereafter. Their brains are dissected, frontal pole, hypothalamus, hippocampus and brain stem dissected and processed for gene expression analysis using microarrays (Affymetrix, USA) and real time quantitative PCR (Taqman; Applied Biosystems, USA).

Chronic Treatment:

On postnatal day (P56) the prenatally stressed and the non-stressed controls are divided into corresponding treatment groups. Their baseline sensory motor gating of is examined using prepulse inhibition (PPI) paradigm. The treatment groups are as follows:

1) Prenatally stressed: Haloperidol p.o. chronic dosing regime.

2) Non-stressed controls: Haloperidol p.o., chronic dosing regime.

3) Prenatally stressed: Clozapine p.o. chronic dosing regime.

4) Non-stressed controls: Clozapine p.o. chronic dosing regime.

5) Prenatally stressed: Fluoxetine p.o. chronic dosing regime.

6) Non-stressed controls: Fluoxetine p.o. chronic dosing regime.

After the treatment, the sensory motor gating is being tested, the animals will be sacrificed, their brains dissected and selected brain regions will be processed for gene expression analysis using microarrays (Affymetrix, USA) or Taqman (Applied Biosystems, USA).

Prenatally stressed animals have been demonstrated previously to have learning and memory deficits (Lemaire et al., Proc. Natl. Acad. Sci. USA 97:11032-11037 (2000)). To further define the cognitive component of the initial gene chip finding, a separate group of animals will be subjected to memory and learning testing (Morris water-maze) and thereafter treated acutely with rivastigmine, methylphenidate and amphetamine. The treatment groups are as follows:

1) Prenatally stressed, rivastigmine i.p.

2) Non-stressed controls, rivastigmine i.p.

3) Prenatally stressed, methylphenidate i.p.

4) Non-stressed controls, methylphenidate i.p.

5) Prenatally stressed, amphetamine i.p.

6) Non-stressed controls, amphetamine i.p.

7) Prenatally stressed, vehicle control i.p.

8) Non-stressed controls, vehicle control i.p.

After the treatment, the cognitive performance is re-assessed. Thereafter, the animals are sacrificed, their brains are dissected and processed for microarray (Affymetrix, USA) and Taqman (Applied Biosystems, USA) analysis of gene expression.

### EXAMPLE 5

### Identification of the Differential Acute Stress Response in the Prenatally Stressed Vs. Prenatally Non-Stressed Frontal Pole Samples

Microarray Analysis

Microarray analysis is performed using GeneSpring® 4.2.1 (Silicon Genetics, Redwood City, Calif., USA) and Novartis Pharmacogenetics Gene Expression Analysis Tools (Novartis proprietary). Microarray quality is first examined and outliers identified based on the percentage of present values, the background noise level and clustering of the individual 48 microarrays using hierarchical clustering of all 8799 genes on the Affymetrix RG U34 A microarray based on Pearson correlation around zero (standard correlation with minimum distance of 0.001 and separation ratio of 0.5). After removal of the outlier microarrays, 25 NS controls and 23 PNS microarrays enter the analysis. For the frontal pole samples (n=5-7 per per each group sacrificed at baseline and 30 min, 150 min and 24 h after the beginning of the acute stress session; total n=48) the background, the percentage of present values, the AFFX-GAPDH3′/AFFX-GAPDH5′ probe expression ratio and the AFFX-GAPDH3′ probe variation are 62.5±9.45, 40.89±3.01, 6.86±7.36 and 23%, respectively. Well above the average variation, a 1.5 fold (=50%) change in the expression signal intensity is chosen to represent a minimum gene expression change resulting from the acute stress exposure (with raw data minimum of 100 and p<0.05 (Welch)).

Data Normalization and Analysis

The expression value for each gene on a chip is divided by the mean of all expression values on that chip assuming that this is at least 10. The bottom tenth percentile is used as a test for correct background subtraction. Thereafter, each gene is normalized to itself by dividing all measurements for that gene by the median of the gene's expression values over all the samples.

Following the normalization, only genes having raw data expression values ≧100 at least in one of the two conditions (NS and PNS) are included in the analysis. Genes showing a minimum fold difference of 1.5 in the average expression signal intensity between the NS and the PNS with a statistical significance of p≦0.005 (Welsch test) are considered as differentially expressed.

The effect of acute stress on the normal, prenatally non-stressed control male offspring (NS), and the prenatally stressed male offspring (PNS) is studied using two analysis approaches (FIG. 1). In the first one, the gene expression changes (raw data expression >100, ≧1.5 fold change, p<0.05) from the baseline to 30 min, 150 min and 24 h following the 30 min acute restraint stress are individually analyzed for the NS and the PNS groups. The differentially expressed gene groups consisting of the acute stress response for the NS or PNS individually are then compared in order to identify a group of common stress response genes and groups of genes that showed a differential expression pattern following the acute stress in either the NS or the PNS group.

In the second approach, gene expression at the baseline and individual timepoints after the acute stress are compared between the NS and PNS animals and differentially expressed genes (raw data expression >100, ≧1.5 fold change, p<0.05) a identified.

TABLE 5
Genes constituting the “normal stress response” after exposure to acute stress.

| | |
|---|---|
| Accession | Description |

Common genes upregulated from baseline to 30 min after acute stress in the normal
controls (prenatally non-stressed) and in prenatally stressed

| | |
|---|---|
| AF030086 | Activity and neurotransmitter-induced early gene 1 (ania-1) |
| AF050659 | Activity and neurotransmitter-induced early gene 7 (ania-7) |
| L01624 | Serum and glucocorticoid-regulated kinase (sgk) |
| L26292 | FSH-regulated protein mRNA |
| M60921 | PC3 NGF-inducible anti-proliferative putative secreted protein (PC3) |
| M65149 | CCAAT/enhancerbinding, protein (C/EBP) delta |
| AA891041 | jun B proto-oncogene |
| AA944156 | B-cell translocation gene 2, anti-proliferative (Btg2) |
| AI175959 | Avian sarcoma virus 17 (v-jun) oncogene homolog (Jun) |
| S81478 | CL100 PTPase = oxidative stress-inducible protein tyrosine phosphatase |
| U02553 | Dual specificity protein phosphatase 1 |
| U17254 | Immediate early gene transcription factor NGFI-B, Nr4a1 |
| U19866 | Growth factor Arc |
| U78102 | krox20 (egr-2) |
| X06769 | c-fos |
| X63594 | RL/IF-1 (IkappaB) |

Common genes upregulated from baseline to 2 h after acute stress

| | |
|---|---|
| D28110 | MOBP (myelin-associated oligodendrocytic basic protein) |

Common genes upregulated by acute stress from baseline to 24 h after acute stress

| | |
|---|---|
| J03179 | D-binding protein |
| AF035955 | Kinesin-related protein KRP6 (KRP6) |

Common genes downregulated by acute stress baseline to 30 min after acute stress

| | |
|---|---|
| X67250 | n-chimaerin |
| AA799531 | ESTs, Weakly similar to M18.3.p [Caenorhabditis elegans] |

Common genes downregulated from baseline to 2 h after acute stress

| | |
|---|---|
| L19699 | Rat GTP-binding protein (ral B) |
| AI639336 | 98.51% zinc finger protein 326 Putative Ortholog (mouse) |
| U04998 | Phosphacan |
| AA892549 | ESTs |
| AA893664 | TEMO (novel protein; PA200 a putative human ortholog) |

Common genes downregulated from baseline to 24 h after acute stress

| | |
|---|---|
| M18416 | Nerve growth factor-induced (NGFI-A, Egr-1) |
| U75397 | Krox-24 (NGFI-A, Egr-1) |
| AF023087 | Nerve growth factor induced factor A (Egr-1, Krox-24) |
| AB003726 | Vesl |

Genes upregulated in the normal controls from baseline to 30 min after acute stress

| | |
|---|---|
| X56325 | 2-alpha-1 globin gene |
| AF020618 | Progression elevated gene 3 |
| X54686 | pJunB |
| AA800680 | ESTs, Weakly similar to S68418 protein phosphatase 1M chain M110 |
| | isoform |
| AA800881 | ESTs, Weakly similar to SUDY |
| AI178971 | Hemoglobin, alpha 1 |
| AA875444 | ESTs, similar to dihydropyriminidase-like 2 |
| AA800245 | ESTs, Weakly similar to T09013 RING finger protein Fxy - mouse |

Genes upregulated in the normal controls from baseline to 2 h after acute stress

| | |
|---|---|
| AA891127 | Nucleolin |
| L01624 | Serum and glucocorticoid-regulated kinase (sgk) |
| U33500 | Retinol dehydrogenase type II |
| AI176456 | ESTs, Highly similar to METALLOTHIONEIN-II |
| C07012 | Peptidylpropyl isomerase C-associated protein |
| AI639415 | ESTs, Weakly similar to KERATIN, TYPE I CYTOSKELETAL 21 |
| D16102 | ATP-stimulated glucocorticoid-receptor translocation promoter |
| U69272 | Interleukin-15 |
| AA800881 | ESTs, Weakly similar to SUDY |

Genes upregulated in the normal controls from baseline to 24 h after acute stress

| | |
|---|---|
| AA893164 | 93.82% expressed sequence AA409659 Putative Ortholog (mouse) |
| X94246 | Pax-8 protein |
| U16253 | Corticotropin-releasing factor receptor subtype 2 (CRF2R) |
| X14674 | Protamine 2 |
| AA800881 | ESTs, Weakly similar to SUDY |
| AA799745 | CDK5 activator-binding protein C53 |
| U33500 | Retinol dehydrogenase type II |
| AA800738 | ESTs, Highly similar to TI60_HUMAN 60 KDA TAT |

Genes downregulated in the normal controls from baseline to 30 min after acute
stress

| | |
|---|---|
| X67250 | R. norvegicus mRNA for n-chimaerin |
| AA799531 | ESTs, Weakly similar to M18.3.p [Caenorhabditis elegans] |
| AA893596 | ESTs, Weakly similar to T43458 hypothetical DKFZp434F0621.1 protein |

Genes downregulated in the normal controls from baseline to 2 h after acute stress

| | |
|---|---|
| L19699 | Rat GTP-binding protein (ral B) |
| AI639336 | 98.51% zinc finger protein 326 Putative Ortholog (mouse) |
| U04998 | Phosphacan |
| AA892549 | ESTs |
| AA893664 | TEMO (novel protein) |

Genes downregulated in the normal controls from baseline to 24 h after acute stress

| | |
|---|---|
| AF036335 | NonO/p54nrb homolog |
| AF063447 | Nuclear RNA helicase |
| AF093267 | Homer-1b |
| J05592 | Protein phosphatase inhibitor-1 protein |
| M15191 | Beta-tachykinin, substance P |
| M32754cd | Inhibin alpha-subunit |
| AA818983 | Diacylglycerol kinase 90 kDa (Dagk) |
| AA875032 | ESTs, hypothetical protein FLJ23306 putative ortholog |
| AA892775 | Lysozyme (lyz) |
| AA892942 | ESTs |
| AI136540 | ESTs, Highly similar to TRT3 RAT TROPONIN T, |
| AI145931 | UDP-N-acetylglucosamine-2-epimerase/N-acetylmannosamine kinase |
| | (uae1) |
| AI176710 | nuclear receptor subfamily 4, group A, member 3 (Nr4a3) |
| S46131 | dopamine D1 receptor {promoter} |
| U10071 | CART protein |
| X95466 | CPG2 protein |

TABLE 6
Genes constituting the “abnormal stress response” after exposure to acute stress.

| | |
|---|---|
| Accession | Description |

Genes upregulated in the prenatally stressed from baseline to 30 min after acute
stress

| | |
|---|---|
| L26292 | FSH-regulated protein mRNA |
| M23697 | Tissue-type plasminogen activator (t-PA) |
| X06769cds | c-fos |
| D28557 | RYB-a |
| AF003835 | Isopentenyl diphosphate-dimethylallyl diphosphate isomerase |
| X76489cds | CD9 Cell surface glycoprotein |
| AF030091 | Activity and neurotransmitter-induced early gene 6 (ania-6) |
| AF030088 | Activity and neurotransmitter-induced early gene 3 (ania-3) |
| M24604 | Proliferating cell nuclear antigen (PCNA/cyclin) |
| X58294 | Carbonic anhydrase II |
| E03358cds | Proteasome (prosome, macropain) subunit, alpha type 2 |
| AA894174 | ESTs, Highly similar to ELECTRON TRANSFER FLAVOPROTEIN |
| | ALPHA-SUBUNIT PRECURSOR |
| U42627 | Dual specificity phosphatase 6 |
| U38180 | Reduced folate carrier membrane glycoprotein |
| U90829 | APP-binding protein 1 |
| M62388 | Ubiquitin conjugating enzyme |
| M12112 | Angiotensinogen |
| M94918 | Beta-globin gene, exons 1-3 |
| AF026529 | Stathmin-like-protein splice variant RB3 |

Genes upregulated in the prenatally stressed from baseline to 2 h after acute stress

| | |
|---|---|
| M93273 | Somatostatin-receptor type 2 |
| AI176710 | Nuclear receptor subfamily 4, group A, member 3 (Nr4a3) |
| X01785 | Rat thymocyte mRNA for cell surface protein (MRC OX-2) |
| X76489cds | CD9 Cell surface glycoprotein |

Genes upregulated in the prenatally stressed from baseline to 24 h after acute stress

| | |
|---|---|
| U20796 | Nuclear receptor Rev-ErbA-beta/Nr1d2 |
| AF078779 | Voltage gated channel like 1 |
| X76489cds | CD9 cell surface glycoprotein |
| AA892500 | 86.89% unc-51-like kinase 2 (C. elegans) |
| AA799721 | ESTs, highly similar to cysteine and histidine rich 1 |
| M31809 | Calcineurin A-beta |
| AA874982 | 97.15% importin beta Putative Ortholog (mouse) |
| AF021350 | Natural killer cell protein group 2-A (NKG2A) |
| AI175959 | Avian sarcoma virus 17 (v-jun) oncogene homolog (Jun) |
| S67769 | Solute carrier family 8 (sodium/calcium exchanger), member 1 |
| AB003992 | SNAP-25B |
| S81478 | PTPase = oxidative stress-inducible protein tyrosine phosphatase |
| U42719 | C4 complement protein |

Genes downregulated in the prenatally stressed from baseline to 30 min after acute
stress

| | |
|---|---|
| X16481 | parathymosin |
| AF064868 | Brain-enriched guanylate kinase-associated protein 1, Begain |
| C07012 | Peptidylpropyl isomerase C-associated protein |
| AI639023 | 89.32% ESTs, Weakly similar to PSF_HUMAN PTB- |
| | ASSOCIATEDSPLICING FACTOR [H. sapiens] |
| X58865mRNA | PFK-L mRNA for liver phosphofructokinase |
| AA799681 | ESTs |
| M88751 | Calcium channel beta subunit-III |
| S70803 | Clone p10.15 product [rats, osteosarcoma ROS17/2.8, mRNA, 737 nt] |
| AA800942 | Complement component 4 (C4a) |
| AI639195 | ESTs |
| AA893749 | EST, Weakly similar to 2206317A protein SS |
| AA893924 | ESTs, Highly similar to KLFD_MOUSE Krueppel-like factor 13 |
| U70270UTR#1 | Mud-4 mRNA |
| Y13714 | Osteonectin |

Genes downregulated in the prenatally stressed from baseline to 2 h after acute
stress

| | |
|---|---|
| L08228exon#22 | N-methyl-D-aspartate receptor (NMDAR1) gene |
| C07012 | Peptidylprolyl isomerase C-associated protein |
| X58865mRNA | PFK-L mRNA for liver phosphofructokinase |
| X17012mRNA | IGFII gene for insulin-like growth factor II |
| AA893924 | ESTs, Highly similar to KLFD_MOUSE Krueppel-like factor 13 |
| M62752 | Statin-like protein |
| AA892053 | ESTs, Highly similar to T42204 chromatin structural protein homolog |
| | Supt5hp —mouse [M. musculus] |
| AF093536 | Beta defensin-1 (BD-1) mRNA |
| U70270UTR#1 | Mud-4 |
| AF065433 | Rattus norvegicus Bcl-2 related ovarian death gene product Bim/BOD |
| AI172476 | TGFB inducible early growth response (Tieg) |
| AF064868 | Brain-enriched guanylate kinase-associated protein 1 (BEGAIN) |
| X60469mRNA | FE65 adaptor protein |
| AI639023 | 89.32% ESTs, Weakly similar to PSF_HUMAN PTB-ASSOCIATED |
| | SPLICING FACTOR |
| L40364 | MHC class I RT1.O type - 149 processed pseudogene |
| D12927 | Transcription elongation factor S-II |
| AI014135 | Beta-carotene 15, 15′-dioxygenase (Bcdo) |

Genes downregulated in the prenatally stressed from baseline to 24 h after acute
stress

| | |
|---|---|
| AF065433 | Bcl-2 related ovarian death gene product Bim/BOD |
| AF093536 | Beta defensin-1 (BD-1) |
| D86711 | Zinc finger, DHHC domain (putative ortholog) |
| M93257 | Cathechol-O-methyltransferase |
| AA800602 | Chemokine (C—X3—C motif) ligand 1, neurotactin |
| AA874843 | CD36 antigen (collagen type I receptor, thrombospondin receptor)-like 1 |
| U49099 | cis-Golgi p28 (p28) |
| U65656 | Matrix metalloproteinase 2 |
| X16481 | Parathymosin |

TABLE 7
Genes significantly differentially expressed at individual timepoints following acute
stress in the prenatally non-stressed (NS)_ and prenatally stressed PNS frontal pole
(“abnormal stress response”).

| | |
|---|---|
| Accession | Description |

Genes upregulated in the PNS group at baseline

| | |
|---|---|
| U66707 | Densin-180 |
| AA900582 | Alpha-2-macroglobulin |
| AI012942 | cell division cycle 25B (cdc25b) |
| AF064868 | Begain |
| AI044716 | Neuronal pentraxin precursor |
| AF068136 | Rattus norvegicus G alpha interacting protein (GAIP) |
| X58865 | PFK-L mRNA for liver phosphofructokinase |
| AI179150 | Cytochrome b oxidase |
| AA800881 | cDNA |
| K02248 | Somatostatin-14 gene |
| AF001423 | N-methyl-D-aspartate receptor NMDAR2A subunit |

Genes downregulated in the PNS group at baseline

| | |
|---|---|
| X01785 | Rat thymocyte mRNA for cell surface protein (MRC OX-2) |
| AA892376 | 93.22% protein associated with PRK1 Putative Ortholog (highly |
| | conserved) |
| AA866459 | ESTs, Highly similar to hypothetical protein MGC4175 [Homo sapiens] |
| AA892483 | 86.36% KIAA0877 protein Putative Ortholog |
| AA891969 | 89.35% nuclear DNA-binding protein Putative Ortholog |
| AJ001641 | Brain-1 (Brn-1) |
| AA799636 | 91.33% ESTs, Highly similar to T00362 hypothetical protein KIAA0675 |
| AA945054 | cytochrome b5 (Cyb5) |
| AI008131 | S-Adenosylmethionine decarboxylase 1A |
| AA893670 | ESTs |
| AI227715 | Retinoblastoma-related gene (Rb2) |
| AA799576 | ESTs, Highly similar to T46259 hypothetical protein |
| | DKFZp761E0323.1 |
| AA799791 | ESTs, Weakly similar to T34021 protein kinase SK2 - rat |
| AA874982 | 97.15% importin beta Putative Ortholog |
| X76489cds | RNCD9 CD9 mRNA for cell surface glycoprotein |
| Y09000 | Dendrin |
| AA859663 | ESTs |
| U77931 | Ribin |
| AI145367 | Adenylyl cyclase-associated protein 2 (Cap2) |
| D10666 | Neural visinin-like protein (NVP) |
| D38560 | CyclinG-associated kinase (Gak) |
| AB003992 | SNAP-25B |
| AA859520 | 97.45% Homo sapiens cDNA FLJ31057 fis, clone HSYRA2000787 |
| AB003991 | SNAP-25A |
| AA894264 | ESTs |

Genes upregulated in the PNS group 30 min after acute stress

| | |
|---|---|
| AF068136 | G alpha interacting protein (GAIP) |
| AI639157 | cDNA clone rx00682 3 |
| AA891901 | Polypyrimidine tract binding protein |
| M30691 | Ly6-C antigen |
| AI234950 | Acid phosphatase 2, lysozymal |
| U66707 | Densin-180 |

Genes upregulated in the PNS group 2 h after acute stress

| | |
|---|---|
| M30691 | Ly6-C antigen |
| X57514 | GABA(A) receptor gamma-1 subunit |
| S79523 | Selectin, lymphocyte membrane protein A.11 (Sell) |
| AI639165 | cDNA clone rx01762 3 |
| D00913 | Intercellular adhesion molecule-1 |
| AJ224680 | Cyclic nucleotide-gated channel beta subunit 1 (Cngp1) |
| AA893711 | ESTs, Weakly similar to DnaJ (Hsp40) homolog, subfamily B, member |
| | 9; Microvascular endothelial differentiation gene 1 |
| AI639422 | ESTs, Moderately similar to CAQC_RAT CALSEQUESTRIN |
| D14015 | Cyclin E |
| L31840 | Nuclear pore complex protein NUP107 |
| AF087674 | Insulin receptor substrate 2 (IRS-2) |
| X05472 | 2.4 kb repeat DNA right terminal region |

Genes downregulated in the PNS group 2 h after acute stress

| | |
|---|---|
| M62752 | Statin-related protein (s1) gene |
| X07729 | Neuron-specific enolase, exons 8-12 |
| AA955388 | ATPase isoform 2, Na+K+ transporting, beta polypeptide 2 |
| AA799479 | ESTs, Moderately similar to NUIM_HUMAN NADH-ubiquinone |
| | oxidoreductase 23 kDa subunit, mitochondrial precursor (Complex I- |
| | 23 KD) (CI-23 KD) (TYKY subunit) |
| H32977 | EST108553 Rattus norvegicus cDNA |
| X54531 | Dynamin-1 |
| AA799791 | ESTs, Weakly similar to T34021 protein kinase SK2 |
| M64986 | Amphoterin |

Genes upregulated in the PNS group 24 h after acute stress

| | |
|---|---|
| AI145494 | Synapsin-2 |
| U66707 | Densin-180 |
| U77583 | Casein kinase I alpha L (CKIaL) |
| K02248 | Somatostatin-14 gene |
| AA892797 | ESTs, Highly similar to A33792 phosphoglycerate kinase (EC 2.7.2.3) - |
| | rat |
| U70268 | mud-7 |
| M88751 | Calcium channel beta subunit-III |

Genes downregulated in the PNS group 24 h after acute stress

| | |
|---|---|
| AA893164 | ESTs |
| AI227715 | Retinoblastoma-related gene Rb2 |
| M35300 | Pancreatic secretory trypsin inhibitor-like protein (PSTI) |
| AB016160 | GABAB receptor 1c, complete cds |
| AI101103 | Vamp2 |
| U77931 | Ribin |
| AI145367 | Adenylyl cyclase-associated protein 2 Cap2 |
| Y09000 | Dendrin |

1-26. (canceled)

27. A method for screening a subject for schizophrenia, bipolar disorder and/or ADHD or a subject at risk of developing schizophrenia, bipolar disorder and/or ADHD, comprising either:
A) detecting a level of expression of at least one gene, selected from the group consisting of (Table 1), in a sample of frontal pole tissue obtained from the subject to provide a first value, with the proviso that if expression of only one gene is detected that the gene is not any of the genes selected from the group consisting of (Table 3); and
comparing the first value with a level of expression of the at least one gene in said detecting step in a sample of frontal pole tissue obtained from a disease-free subject, wherein a greater expression level in the subject sample compared to the sample from the disease-free subject of at least one gene with accession number X57514, U66707, AI639165, AF064868, or AI145494 or wherein a lower expression level in the subject sample compared to the sample from the disease-free subject of at least one gene with accession number U20643, H31232, S61973, AB016160, X85184, J04063, S81353, M33025, AA858621, M74494, AA866358, M64986, AI227715, U77931, AA89392, AA859633, AA891969, AI145367, AA893711, Y09000, AA891940, D26564, AI101103, or AF014009 is indicative of the subject having schizophrenia, bipolar disorder and/or ADHD or at risk of developing schizophrenia, bipolar disorder and/or ADHD; or
B) detecting a level of expression of at least one gene, selected from the group consisting of (Table 2), in a sample of hypothalamus tissue obtained from the subject to provide a first value, with the proviso that if expression of only one gene is detected that the gene is not any of the genes selected from the group consisting of (Table 3); and
comparing the first value with a level of expression of the at least one gene in said detecting step in a sample of hypothalamus tissue obtained from a disease-free subject, wherein a greater expression level in the subject sample compared to the sample from the disease-free subject of at least one gene with accession number AA946532, AB016160, U57500, L17127, M28648, L24776, AB020504, U77931, U75927, AF096269, AA892801, U49099, AA859597, AA875427, AF028784, L22760, AI014135, AI104513, AA894148, AI073204 or D38468, wherein a lower expression level in the subject sample compared to the sample from the disease-free subject of at least one gene with accession number M13100, M59980, M18331, M36418, X57764, U62897, S71570, M16112, AI008131, X12744, AI230260, U48245, U66707, H31692, D89863, X06564, M91234, U31554, AA875659, AA799421, AI014091, AB012234, U09793, AA800513, AI639381, AA799515, X83546, AI013194, AB008538, X52817, AA893065, H31588, AA859832, AI008074, AA851749, AA894321, AI227608, E13644, AA925762, M72422, M17526, U45479, U86635, AA894089, or AA891069 is indicative of the subject having schizophrenia, bipolar disorder and/or ADHD or at risk of developing schizophrenia, bipolar disorder and/or ADHD; or
C) detecting a level of expression of at least one gene, selected from the group consisting of (Table 7), in a sample of frontal pole tissue obtained from the subject to provide a first value, with the proviso that if expression of only one gene is detected that the gene is not any of the genes selected from the group consisting of (Table 3); and
comparing the first value with a level of expression of the at least one gene in said detecting step in a sample of frontal pole tissue obtained from a disease-free subject, wherein a greater expression level in the subject sample compared to the sample from the disease-free subject of at least one gene with accession number U66707, AA900582, AI012942, AF064868, AI044716, AF068136, X58865, AI179150, AA800881, K02248, AF001423, AI639157, AA891901, M30691, AI234950, X57514, S79523, AI639165, D00913, AJ224680, AA893711, AI639422, D14015, L31840, AF087674, X05472, AI145494, U77583, AA892797, U70268 or M88751 or wherein a lower expression level in the subject sample compared to the sample from the disease-free subject of at least one gene with accession number AA892376, AA866459, AA892483, AA891969, AJ001 641, AA799636, AA945054, AI008131, AA893670, AI227715, AA799576, AA799791, AA874982, X76489cds, Y09000, AA859663, U77931, AI145367, D10666, D38560, AB003992, AA859520, AB003991, AA894264, AA955388, X07729, M62752, AA799479, H32977, X54531, M64986, AA893164, M35300, AB016160 or AI101103 is indicative of the subject having schizophrenia, bipolar disorder and/or ADHD or at risk of developing schizophrenia, bipolar disorder and/or ADHD.

28. The method of claim 27, wherein the level of expression of at least two genes, selected from the group consisting of (Tables 1, 2 and 7), are detected.

29. The method of claim 27 (A) or (B), wherein the at least one gene is selected from the group consisting of genes with accession numbers: U20643, H31232, S61973, AB016160, X85184, J04063, S81353, M33025, X57514, AA858621, M74494, M13100, M59980, AA946532, M18331, M36418, X57764, U62897, U57500, L17127, S71570, M16112, AI008131, M28648, X12744, AI230260, U86635, and D38468.

30. The method of claim 27, wherein the level of expression of the gene is determined by detecting the level of expression of a mRNA corresponding to the gene.

31. The method of claim 30, wherein the level of expression of mRNA is detected by techniques selected from the group consisting of Microarray analysis, Northern blot analysis, reverse transcription PCR, and real time quantitative PCR.

32. The method of claim 27, wherein the level of expression of the gene is determined by detecting the level of expression of a protein encoded by the gene.

33. A method for monitoring the progression of schizophrenia, bipolar disorder, and/or ADHD in a subject having, or at risk of having, schizophrenia, bipolar disorder, and/or ADHD, comprising either:
A) measuring a level of expression of at least one gene, selected from the group consisting of (Table 1), over time in the frontal pole tissue sample obtained from the subject with the proviso that if expression of only one gene is detected that the gene is not any of the genes selected from the group consisting of (Table 3), wherein an increase in the level of expression of the at least one gene with accession number: X57514, U66707, AI639165, AF064868, or AI145494 over time is indicative of the progression of schizophrenia, bipolar disorder, and/or ADHD in the subject, or wherein a decrease in the level of expression of the at least one gene with accession number U20643, H31232, S61973, AB016160, X85184, J04063, S81353, M33025, AA858621, M74494, AA866358, M64986, AI227715, U77931, AA89392, AA859633, AA891969, AI145367, AA893711, Y09000, AA891940, D26564, AI01103, or AF014009 over time is indicative of the progression of schizophrenia, bipolar disorder, and/or ADHD in the subject; or
B) measuring a level of expression of at least one gene, selected from the group consisting of (Table 2), over time in the hypothalamus tissue sample obtained from the subject with the proviso that if expression of only one gene is detected that the gene is not any of the genes selected from the group consisting of (Table 3), wherein an increase in the level of expression of the at least one gene with accession number AA946532, AB016160, U57500, L17127, M28648, L24776, AB020504, U77931, U75927, AF096269, AA892801, U49099, AA859597, M875427, AF028784, L22760, AI014135, AI104513, M894148, AI073204 or D38468 over time is indicative of the progression of schizophrenia, bipolar disorder, and/or ADHD in the subject, or wherein a decrease in the level of expression of the at least one gene with accession number M13100, M59980, M18331, M36418, X57764, U62897, S71570, M16112, AI008131, X12744, AI230260, U48245, U66707, H31692, D89863, X06564, M91234, U31554, M875659, AA799421, AI014091, AB012234, U09793, M800513, AI639381, AA799515, X83546, AI013194, AB008538, X52817, AA893065, H31588, AA859832, AI008074, AA851749, AA894321, AI227608, E13644, AA925762, M72422, M17526, U45479, U86635, AA894089, or AA891069 over time is indicative of the progression of schizophrenia, bipolar disorder, and/or ADHD in the subject.

34. The method of claim 33, wherein the level of expression of at least two genes, selected from the group consisting of (Table 1 and 2), are measured.

35. The method of claim 33, wherein the at least one gene is selected from the group consisting of the genes with accession number U20643, H31232, S61973, AB016160, X85184, J04063, S81353, M33825, X57514, AA858621, M74494, M13100, M59980, AA946532, M18331, M36418, X57764, U62897, U57500, L17127, S71570, M16112, AI008131, M28648, X12744, AI230260, U86635, and D38468.

36. A method for identifying agents for use in the treatment of schizophrenia, bipolar disorder, and for ADHD, comprising either:
A.
a) contacting a sample of cells expressing at least one gene selected from the group consisting of (Tables 1 and 2) with a candidate agent;
b) detecting a level of expression of at least one gene in said cells, with the proviso that if expression of only one gene is detected that the gene is not any of the genes selected from the group consisting of (Table 3); and
c) comparing the level of expression of the at least one gene in the sample in the presence of the candidate agent with a level of expression of the at least one gene in cells that are not contacted with the candidate agent, wherein for genes with accession number U20643, H31232, S61973, X85184, J04063, S81353, M33025, AA858621, M74494, AA866358, M64986, AI227715, AA89392, AA859633, AA891969, AI145367, AA893711, Y09000, AA891940, D26564, AI101103, AF014009, M13100, M59980, M18331, M36418, X57764, U62897, S71570, M16112, AI008131, X12744, AI230260, U48245, H31692, D89863, X06564, M91234, U31554, AA875659, AA799421, AI014091, AB012234, U09793, AA800513, AI639381, AA799515, X83546, AI013194, AB008538, X52817, AA893065, H31588, AA859832, AI008074, AA851749, AA894321, AI227608, E13644, AA925762, M72422, M17526, U45479, U86635, AA894089, or AA891069 an increased level of expression of the at least one gene in the sample in the presence of the candidate agent relative to the level of expression of the at least one gene in the sample in the absence of the candidate agent, or wherein for genes with accession number X57514, AI639165, AF064868, AI145494, AA946532, U57500, L17127, M28648, L24776, AB020504, U75927, AF096269, AA892801, U49099, AA859597, AA875427, AF028784, L22760, AI014135, AI104513, AA894148, AI073204 or D38468 a decreased level of expression of the at least one gene in the sample in the presence of the candidate agent relative to the level of expression of the at least one gene in the sample in the absence of the candidate agent and for genes with accession numbers AB016160 and U77931 an increased level of expression with respect to the frontal pole, and a decreased expression with respect to the hypothalamus and for a gene with accession number U66707 an increased expression with respect to hypothalamus and an decreased expression with respect to the frontal pole is indicative of an agent useful in the treatment of schizophrenia, bipolar disorder, and/or ADHD; or
B.
a) contacting a sample of cells expressing at least one gene, selected from the group consisting of (Table 7) with a candidate agent;
b) detecting level of expression of at least one gene in said cells with the proviso that if expression of only one gene is detected that the gene is not any of the genes selected from the group consisting of (Table 3); and
c) comparing the level of expression of the at least one gene in the sample in the presence of the candidate agent with a level of expression of the at least one gene in cells that are not contacted with the candidate agent, wherein for genes with accession number: AA892376, AA866459, AA892483, AA891969, AJ001641, AA799636, AA945054, AI008131, AA893670, AI227715, AA799576, AA799791, AA874982, X76489cds, Y09000, AA859663, U77931, AI145367, D10666, D38560, AB003992, AA859520, AB003991, AA894264, AA955388, X07729, M62752, AA799479, H32977, X54531, M64986, AA893164, M35300, AB016160 or AI101103 an increased level of expression of the at least one gene in the sample in the presence of the candidate agent relative to the level of expression of the at least one gene in the sample in the absence of the candidate agent: or wherein for genes with accession number: U66707, AA900582, AI012942, AF064868, AI044716, AF068136, X58865, AI79150, AA800881, K02248, AF001423, AI639157, AA891901, M30691, AI234950, X57514, S79523, AI639165, D00913, AJ224680, AA893711, AI639422, D14015, L31840, AF087674, X05472, AI145494, U77583, AA892797, U70268 or M88751 a decreased level of expression of the at least one gene in the sample in the presence of the candidate agent relative to the level of expression of the at least one gene in the sample in the absence of the candidate agent; is indicative of an agent useful in the treatment of schizophrenia, bipolar disorder, and/or ADHD.

37. The method of claim 36, wherein the level of expression of at least two genes in the sample is detected the detecting step (b).

38. The method of claim 36(A), wherein the at least one gene is selected from the group consisting of genes with accession number U20643, H31232, S61973, AB016160, X85184, J04063, S81353, M33025, X57514, AA858621, M74494, M13100, M59980, AA946532, M18331, M36418, X57764, U62897, U57500, L17127, S71570, M16112, AI008131, M28648, X12744, AI230260, U86635, and D38468.

39. A method of treating and preventing schizophrenia, bipolar disorder, and/or ADHD in patient in need thereof, comprising either:
A) administering to said patient an effective amount of an agent that can induce a decrease in the expression of at least one gene with accession number U66707, AA900582, AI012942, AF064868, AI044716, AF068136, X58865, AI179150, AA800881, K02248, AF001423, AI639157, AA891901, M30691, AI234950, X57514, S79523, AI639165, D00913, AJ224680, AA893711, AI639422, D14015, L31840, AF087674, X05472, AI145494, U77583, AA892797, U70268 and/or M88751; and/or induce an increase of at least one gene with accession number AA892376, AA866459, AA892483, AA891969, AJ001641, AA799636, AA945054, AI008131, AA893670, AI227715, AA799576, AA799791, AA874982, X76489cds, Y09000, AA859663, U77931, AI145367, D10666, D38560, AB003992, AA859520, AB003991, AA894264, AA955388, X07729, M62752, AA799479, H32977, X54531, M64986, AA893164, M35300, AB016160 and/or for AI101103, with the proviso that if expression of only one gene is altered that the gene is not any selected from the group consisting of (Table 3); or
B) administering to said patient an effective amount of an agent that can induce a decrease in the expression of at least one gene with accession number X57514, AI639165, AF064868, AI145494, AA946532, U57500, L17127, L24776, A8020504, U75927, AF096269, AA892801, U49099, AA859597, AA875427, AF028784, L22760, AI014135, AI104513, AA894148, AI073204 and/or D38468; and/or induce an increase of at least one gene with accession number U20643, H31232, S61973, X85184, J04063, S81353, M33025, AA858621, M74494, AA866358, M64986, AI227715, AA89392, AA859633, AA891969, AI145367, AA893711, Y09000, AA891940, D26564, AI101103, M28648, AF014009, M13100, M59980, M18331, M36418, X57764, U62897, S71570, M16112, AI008131, X12744, AI230260, U48245, H31692, D89863, X06564, M91234, U31554, AA875659, AA799421, AI014091, AB012234, U09793, AA800513, AI639381, AA799515, X83546, AI03194, AB008538, X52817, AA893065, H31588, AA859832, AI008074, AA851749, AA894321, AI227608, E13644, AA925762, M72422, M17526, U45479, U86635, AA894089, and/or AA891069; and/or for genes AB016160 and/or U7793-lan increase with respect to frontal pole expression and a decrease to the hypothalamus expression; and/or for the gene U66707 a decrease with respect to the frontal pole and an increase with respect to the hypothalamus, with the proviso that if expression of only one gene is altered that the gene is not any gene of selected from the group consisting of (Table 3).

40. A method for monitoring the efficacy of a treatment of a subject having schizophrenia, bipolar disorder, and/or ADHD or at risk of developing schizophrenia, bipolar disorder, and/or ADHD with an agent, comprising either:
A.
a) obtaining a pre-administration sample in the frontal lobe from the subject prior to administration of the agent;
b) detecting a level of expression of at least one gene, selected from the group consisting of (Tables 1 and 7), in the pre-administration sample, with the proviso that if expression of only one gene is detected that the gene is not any gene selected from the group consisting of (Table 3);
c) obtaining one or more post-administration samples from the subject;
d) detecting a level of expression of the at least one gene in the post-administration sample or samples;
e) comparing the level of expression of the at least one gene in the pre-administration sample with the level of expression of the at least one gene in the post-administration sample; and
f) adjusting the administration of the agent accordingly; or
B.
a) obtaining a pre-administration sample in the hypothalamus from the subject prior to administration of the agent;
b) detecting a level of expression of at least one gene, selected from the group consisting of (Table 2), in the pre-administration sample, with the proviso that if expression of only one gene is detected that the gene is not any gene selected from the group consisting of (Table 3);
c) obtaining one or more post-administration samples from the subject;
d) detecting a level of expression of the at least one gene in the post-administration sample or samples;
e) comparing the level of expression of the at least one gene in the pre-administration sample with the level of expression of the at least one gene in the post-administration sample; and
f) adjusting the administration of the agent accordingly.

41. The method of claim 40, wherein the level of expression of at least two genes, selected from the group consisting of (Tables 1, 2 and 7), is detected in step (b).

42. The method of claim 40, wherein the level of expression of mRNA is detected by techniques selected from the group consisting of Microarray analysis, Northern blot analysis, reverse transcription PCR and real time quantitative PCR.

43. A transgenic mouse whose genome comprises a disruption of any of the endogenous genes selected from the group consisting of (Table 1, 2 and 7), wherein said disruption comprises the insertion of a transgene, and wherein said disruption results in said transgenic mouse not exhibiting normal expression of any of said endogenous genes with the proviso that if only one gene is disrupted it is not one of the genes of selected from the group consisting of (Table 3).

44. A method for producing a rodent having schizophrenia, bipolar disorder, and/or ADHD or at risk of developing schizophrenia, bipolar disorder, and/or ADHD, comprising subjecting the rodent to variable repeated stress during the last week of the gestation, starting on embryonic day 14 and continuing until the natural delivery of a pup at about embryonic day 22.
